SEQUENCE LISTING (98 sequences)

SEQ ID NO: 1 - DNA, 1401 bp, Artificial sequence
  Synthetic
atggacaagaagcaagtgaccgatctgcgcagcgagctgctggactcgcggttcggcgccaagtccatcagcacgatcgcggagtcgaagcggttcccgctccacgagatgcgcgacgacgtggcgttccagatcatcaatgacgagctgtacctggacggcaacgcccgccagaacctggccaccttctgccagacgtgggacgatgaaaacgtgcataagctgatggacctgagcattaacaagaactggatcgacaaggaagagtacccgcagagcgcggccatcgacctccgctgcgtcaacatggtggccgatctgtggcacgccccggccccgaagaatggccaggccgtgggcaccaacaccatcggcagcagcgaagcctgcatgctgggcggcatggccatgaagtggcgctggcgcaagcgcatggaagcggcgggcaagccgaccgacaagccgaacctggtctgcggccccgtgcaaatctgctggcacaagtttgcgcgttactgggacgtggagctgcgggaaatcccgatgcgcccgggccagctgttcatggacccgaagcgcatgatcgaggcctgcgacgagaacacgatcggcgtggtgccgacgttcggcgtgacctacacgggcaattacgagttcccgcagcccctgcacgacgcgctggacaagttccaggccgatacgggcatcgatatcgacatgcacatcgacgcggcctcgggcggtttcctggcccccttcgtcgcccccgacatcgtctgggacttccgcctgccccgcgtcaagtcgatctcggcgtcgggccataagttcggcctggcgccgctcggttgcggctgggtcatctggcgcgacgaagaagcgctgccgcaagagctggtgttcaacgtggactacctgggcggccagatcggcaccttcgccattaacttctcccggcccgccggccaggtgatcgcgcagtactacgagtttctgcgcctgggtcgcgagggctacaccaaggtgcagaacgcctcctatcaggtcgcggcgtatctggccgacgagatcgccaagctcggcccctacgagttcatctgcaccggccgtccggatgaaggcatcccggccgtctgcttcaagctgaaggacggcgaggacccgggctacaccctgtacgacctgtcggaacgcctgcgcctgcgtggctggcaagtccccgccttcaccctgggtggcgaggccaccgatatcgtcgtgatgcgcattatgtgccgccgcggctttgaaatggacttcgcggaactgctcctggaagattataaggcctcgctcaagtatctctccgaccacccgaagctccagggcatcgcgcagcagaacagcttcaagcatacgtga

SEQ ID NO: 2 - protein, 465 aa, E. coli
MDKKQVTDLRSELLDSRFGAKSISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKH

SEQ ID NO: 3 - DNA, 1404 bp, Artificial Sequence
  Synthetic
atgccccagtggcacccgcaccgcgagcagaagaacctccccgacgagttccccgtgaacccgctgttctcgcgccagggcgaagtcaccattccgcgcctgcgcatcggcgaccagggcatgctgccggaaacggcgtaccagatcatccacgacgagatcgccctggacggcaatgcccggctgaacctggcgaccttcgtcaccacctggatggagcccgacgcgaagcgcctgtacggcgagtccttcgacaagaacatgatcgacaaggatgagtatccgcagaccgccgccatcgaggaacgctgcgtgcgcatcctcgcggacctgtggaacagcccgaatccggacaccacgatgggcgtgagcaccacgggctccagcgaggcctgcatgctgggcggcctggccctgaagcgccggtggcagaagctgcggaagtcgaagggtctgagcaccgaccgccccaacatcgtctttagctcgtcggtccaggtcgtgtgggagaagtttgcgaactactgggacgtggaaccgcgctacgtcaatatcaaccccgaccacccgtacctggatgcggaaggcgtgatcaacgccgtggacgaaaacacgatcggcgtggtgccgatcctgggcgtcacgtataccggtggctacgagccgatcgccgcgatcgccaaggccctggacgaactgcaggaaaagaccggcctggacatcccgattcacgtggacgccgcgtcgggcggcttcatcgccccgttcctgcagccggatctgatctgggacttccgtctgccgcgtgtgaagtccattaacgtgtcgggccataagtatggtctggtctaccccggcctgggctgggtcatctggcgcgaaaaggaggatctccccgaggacctgatcttccgcgtgtcctacctcggcggcaacatgcccaccttcgcgctgaacttctcgcgccccggcgcccaggtcctgctgcagtactataacttcctgcgcctgggcaaggacggctactacgcggtgcaaaagaccagccaggaaaatgccctgttcctcagcaaagaaatcggcgagatggacgcgtttgagatcctggccgacggctccgacatcccggtgctcgcctggaagctgaaggaagattataccccgaattggacgctgtacgacctctcgcggcagctccgcacgtacggctggcaagtgccggcctacccgctgccggcggacatggaagagatcaccatcatgcgcatcgtggtccgcaacggcttcagccgtgacctggcgcacctgttcatggtcaacttcaagcaagccgtggagttcctgaactcgctcgatcgcccggtgctgaaggataccaagtacgacaacggcttccatcattga

SEQ ID NO: 4 - protein, 467 aa, B. megaterium
MPQWHPHREQKNLPDEFPVNPLFSRQGEVTIPRLRIGDQGMLPETAYQIIHDEIALDGNARLNLATFVTTWMEPDAKRLYGESFDKNMIDKDEYPQTAAIEERCVRILADLWNSPNPDTTMGVSTTGSSEACMLGGLALKRRWQKLRKSKGLSTDRPNIVFSSSVQVVWEKFANYWDVEPRYVNINPDHPYLDAEGVINAVDENTIGVVPILGVTYTGGYEPIAAIAKALDELQEKTGLDIPIHVDAASGGFIAPFLQPDLIWDFRLPRVKSINVSGHKYGLVYPGLGWVIWREKEDLPEDLIFRVSYLGGNMPTFALNFSRPGAQVLLQYYNFLRLGKDGYYAVQKTSQENALFLSKEIGEMDAFEILADGSDIPVLAWKLKEDYTPNWTLYDLSRQLRTYGWQVPAYPLPADMEEITIMRIVVRNGFSRDLAHLFMVNFKQAVEFLNSLDRPVLKDTKYDNGFHH

SEQ ID NO: 5 - DNA, 5899 bp, Artificial Sequence
  Synthetic
atgctgtacgacaagtcgctggaacgcgacaactgcggcttcggcctcattgcgcacatcgaaggcgaaccgagccataaggtcgtccgcacggcgatccacgcgctcgcgcgtatgcagcaccgcggtgccatcctggcggacggcaagaccggtgatggctgcggtctgctcctgcagaagcccgaccgcttcttccgcatcgtggcccaagagcgcggctggcgcctggcgaagaactacgccgtgggcatgctgttcctgaacaaggacccggaactggccgcggcggcccgccgtatcgtggaagaggaactgcagcgcgaaaccctgagcatcgtcggctggcgcgacgtgcccaccaacgaaggcgtgctcggcgaaatcgccctgtcctcgctgccccggatcgagcagattttcgtgaacgcgcccgccggctggcgcccgcgcgacatggagcggcgcctctttatcgcccgccggcgcatcgagaagcgcctggaggccgacaaggatttctacgtgtgcagcctgtcgaacctcgtgaacatttacaagggcctgtgcatgccgaccgacctcccgcgcttctacctggacctggcggacctccggctggagtcggcgatctgcctgttccatcagcgcttttcgaccaacaccgtgccgcgctggccgctcgcccagccgttccgctacctcgcccacaacggcgaaatcaacacgatcaccggcaaccgccagtgggcccgggcccgcacctacaagtttcagacgccgctgatcccggacctccacgacgccgcgccgttcgtgaacgaaaccggcagcgactcctcgtcgatggacaacatgctggagctgctgctcgccggcggcatggacatcatccgcgcgatgcgcctgctggtgccccccgcctggcagaataacccggacatggacccggaactgcgggccttcttcgacttcaacagcatgcacatggagccctgggacggcccggccggcatcgtgatgagcgacggtcgcttcgccgcgtgcaacctggaccggaacggcctgcgcccggcgcgctacgtgatcacgaaggataagctgatcacgtgcgcctccgaagtcggcatctgggattaccagccggacgaagtggtcgagaagggccgcgtgggtccgggcgaactgatggtgatcgacacgcgctcgggccgcatcctgcacagcgccgaaaccgacgacgacctgaagtcccgccacccgtacaaggaatggatggagaagaatgtgcgccgcctggtcccgtttgaggacctcccggacgaagaggtgggctcgcgcgagctggatgacgacacgctggccagctaccagaagcagttcaattactcggcggaagaactggatagcgtcatccgcgtgctgggtgagaacggtcaagaagccgtcggctcgatgggcgatgacaccccgttcgccgtgctgtccagccagccccgcatcatctacgactacttccgccagcagtttgcccaagtcaccaatccgccgatcgacccgctgcgcgaagcccatgtcatgagcctcgcgacctcgatcggtcgcgagatgaacgtgttctgcgaagccgagggccaagcgcaccgcctgagcttcaagagcccgatcctgctgtattccgatttcaagcaactgacgacgatgaaggaagaacattatcgcgccgacaccctcgacatcacgttcgatgtcaccaagacgaccctggaggccaccgtgaaggaactctgcgacaaggccgaaaagatggtccgcagcggcaccgtgctgctcgtgctgtcggaccgcaacatcgccaaggaccgtctgccggtgccggccccgatggcggtgggcgccatccagacccgcctggtggaccagtcgctgcgctgcgacgcgaacatcatcgtggaaacggcgagcgcccgggacccccaccactttgccgtcctgctgggcttcggcgccacggccatctacccctatctggcctacgaaacgctcggccgcctggtggacacgcatgccatcgcgaaggactaccgcaccgtgatgctgaactatcggaacggcatcaacaagggcctgtacaagatcatgtcgaagatgggcatctcgaccatcgcctcgtatcgctgctcgaagctgttcgaggccgtcggtctgcatgacgatgtcgtcggcctgtgcttccagggtgcggtgtcccgtatcggcggcgcctccttcgaggacttccagcaggatctgctgaacctgagcaagcgcgcctggctggcgcgcaagccgatcagccagggcggcctcctgaagtacgtgcatggcggcgagtaccacgcctataaccccgatgtcgtgcgcacgctgcaacaggcggtccagagcggcgaatattcggattatcaagagtacgcgaagctggtgaacgaacgcccggcgaccaccctgcgcgacctcctggccattaccccgggcgagaacgccgtgaacatcgcggacgtggaaccggccagcgaactcttcaagcgcttcgacacggcggcgatgtcgatcggcgcgctgtcgccggaggcgcacgaagcgctggccgaggcgatgaacagcattggcggcaactccaactcgggcgagggcggcgaagatccggcgcgctacggcaccaacaaggtcagccggatcaagcaagtggcgagcggccgtttcggcgtgacgcccgcgtatctggtgaacgccgacgtgatccagatcaaggtggcccagggcgccaagccgggtgagggcggccaactgcccggcgataaggtcacgccgtatattgccaagctgcgctacagcgtgcccggcgtgaccctgatctcccccccgccccatcacgacatctattcgatcgaggacctggcccagctgatcttcgacctgaagcaagtgaaccccaaggccatgatttccgtcaagctcgtgtccgagcccggcgtcggcaccatcgccacgggcgtggcgaaggcctacgccgacctgatcaccatcgcgggctacgacggcggcaccggcgcgtcgccgctgagcagcgtgaagtacgccggctgcccctgggagctgggcctcgtggaaacccaacaggccctggtcgccaacggcctccggcacaagatccgcctgcaagtggacggcggcctcaagacgggcgtggatatcattaaggccgccatcctgggcgcggagagcttcggcttcggcacgggcccgatggtggcgctgggctgcaagtacctccgcatctgccacctgaacaactgcgccaccggcgtcgccacccaagatgacaagctgcgtaagaaccactatcatggcctgccgttcaaggtcacgaactatttcgagtttattgcccgggaaacgcgcgaactgatggcccagctcggcgtcacgcgcctggtggacctgatcggccgcacggacctcctgaaggaactggacggtttcacggccaagcagcagaagctggccctgtcgaagctgctggaaacggccgagccgcatcccggcaaggccctgtactgcaccgagaacaacccgcccttcgacaacggcctgctgaatgcccagctgctgcagcaggccaagccgtttgtggacgagcgccagtccaagaccttctggttcgacatccgcaataccgaccggtccgtgggcgcctccctgtcgggctatatcgcccagacccacggtgatcagggcctggcggccgacccgatcaaggcctatttcaatggcaccgccggccagtcgttcggtgtctggaacgcgggcggcgtcgagctgtacctgaccggcgacgccaacgactacgtgggcaagggtatggccggcggcctgatcgccatccgccccccggtgggctcggcgttccgttcccacgaagcgtcgatcatcggcaatacgtgcctgtacggcgcgaccggcggtcgcctgtacgccgccggccgcgcgggcgaacgcttcggcgtccgcaactcgggcgccatcacggtggtggagggcatcggcgacaacggctgcgagtacatgaccggtggcatcgtctgcatcctgggcaagaccggcgtcaatttcggcgcgggtatgacgggcggcttcgcgtacgtcctggatgagtccggcgacttccgcaagcgcgtcaacccggagctggtcgaggtgctgtcggtggacgccctggcgatccatgaggagcacctccgcggcctgatcacggagcacgtgcagcacaccggctcgcagcggggtgaagagatcctcgccaactggagcaccttcgcgacgaagttcgcgctggtcaagccgaagtcgtcggatgtgaaggccctgctgggccatcgctcgcgcagcgccgccgaactgcgcgtgcaggcccaatgaggataaaggaggtatatcgatgtcccaaaacgtgtatcagttcatcgacctccagcgcgtggacccgccgaagaagccgctgaagatccgtaagatcgagttcgtggaaatctacgagcccttcagcgagggccaggccaaggcccaggccgaccgctgcctgtcgtgcggcaacccgtactgcgagtggaagtgcccggtgcataactacatcccgaattggctgaagctcgcgaacgaaggccgcatcttcgaagcggccgagctgtcgcatcagacgaataccctgcccgaggtgtgcggccgggtgtgcccgcaagaccgcctgtgcgagggctcctgcaccctgaacgacgagttcggcgccgtgacgatcggcaatatcgaacgctatatcaacgacaaggcctttgaaatgggttggcgtcccgatatgtcgggcgtgaagcagacgggcaagaaggtcgcgatcatcggcgccggcccggccggcctggcgtgcgccgacgtgctgacgcggaacggcgtcaaggccgtggtgttcgaccgccaccccgagatcggtggcctgctgacgttcggcatcccggcgttcaagctggaaaaggaagtcatgacgcgccgccgggaaatcttcaccggcatgggcatcgagttcaagctgaacaccgaagtcggccgcgacgtgcagctggacgatctgctgagcgactacgacgcggtgttcctgggcgtcggcacctaccagagcatgcgcggcggcctggagaatgaagatgcggacggcgtgtacgccgcgctgccgttcctcatcgcgaacaccaagcaactcatgggcttcggcgaaacccgcgacgagccgttcgtcagcatggaaggcaagcgcgtggtcgtgctcggtggcggcgacaccgcgatggactgcgtccgcaccagcgtgcgccagggcgccaagcacgtcacgtgcgcctaccgccgcgatgaagaaaacatgccgggctcgcgccgcgaggtgaagaacgcgcgggaagagggcgtggagtttaagttcaacgtccagcccctgggcatcgaagtgaacggcaacggtaaggtgtcgggcgtcaagatggtccgcaccgagatgggcgaaccggacgccaagggccgtcgccgcgcggagattgtcgccggctccgagcacatcgtgcccgccgatgccgtgatcatggccttcggcttccgtccgcacaacatggagtggctggcgaagcactcggtggagctggacagccagggccggatcatcgcccccgagggctccgacaacgcgtttcagacctcgaacccgaagattttcgcgggcggcgatatcgtgcgcggctcggacctggtcgtcaccgcgatcgccgagggtcgcaaggccgccgacggtatcatgaattggctggaagtgtga

SEQ ID NO: 6 - protein, 1486 aa, E. coli
MLYDKSLERDNCGFGLIAHIEGEPSHKVVRTAIHALARMQHRGAILADGKTGDGCGLLLQKPDRFFRIVAQERGWRLAKNYAVGMLFLNKDPELAAAARRIVEEELQRETLSIVGWRDVPTNEGVLGEIALSSLPRIEQIFVNAPAGWRPRDMERRLFIARRRIEKRLEADKDFYVCSLSNLVNIYKGLCMPTDLPRFYLDLADLRLESAICLFHQRFSTNTVPRWPLAQPFRYLAHNGEINTITGNRQWARARTYKFQTPLIPDLHDAAPFVNETGSDSSSMDNMLELLLAGGMDIIRAMRLLVPPAWQNNPDMDPELRAFFDFNSMHMEPWDGPAGIVMSDGRFAACNLDRNGLRPARYVITKDKLITCASEVGIWDYQPDEVVEKGRVGPGELMVIDTRSGRILHSAETDDDLKSRHPYKEWMEKNVRRLVPFEDLPDEEVGSRELDDDTLASYQKQFNYSAEELDSVIRVLGENGQEAVGSMGDDTPFAVLSSQPRIIYDYFRQQFAQVTNPPIDPLREAHVMSLATSIGREMNVFCEAEGQAHRLSFKSPILLYSDFKQLTTMKEEHYRADTLDITFDVTKTTLEATVKELCDKAEKMVRSGTVLLVLSDRNIAKDRLPVPAPMAVGAIQTRLVDQSLRCDANIIVETASARDPHHFAVLLGFGATAIYPYLAYETLGRLVDTHAIAKDYRTVMLNYRNGINKGLYKIMSKMGISTIASYRCSKLFEAVGLHDDVVGLCFQGAVSRIGGASFEDFQQDLLNLSKRAWLARKPISQGGLLKYVHGGEYHAYNPDVVRTLQQAVQSGEYSDYQEYAKLVNERPATTLRDLLAITPGENAVNIADVEPASELFKRFDTAAMSIGALSPEAHEALAEAMNSIGGNSNSGEGGEDPARYGTNKVSRIKQVASGRFGVTPAYLVNADVIQIKVAQGAKPGEGGQLPGDKVTPYIAKLRYSVPGVTLISPPPHHDIYSIEDLAQLIFDLKQVNPKAMISVKLVSEPGVGTIATGVAKAYADLITIAGYDGGTGASPLSSVKYAGCPWELGLVETQQALVANGLRHKIRLQVDGGLKTGVDIIKAAILGAESFGFGTGPMVALGCKYLRICHLNNCATGVATQDDKLRKNHYHGLPFKVTNYFEFIARETRELMAQLGVTRLVDLIGRTDLLKELDGFTAKQQKLALSKLLETAEPHPGKALYCTENNPPFDNGLLNAQLLQQAKPFVDERQSKTFWFDIRNTDRSVGASLSGYIAQTHGDQGLAADPIKAYFNGTAGQSFGVWNAGGVELYLTGDANDYVGKGMAGGLIAIRPPVGSAFRSHEASIIGNTCLYGATGGRLYAAGRAGERFGVRNSGAITVVEGIGDNGCEYMTGGIVCILGKTGVNFGAGMTGGFAYVLDESGDFRKRVNPELVEVLSVDALAIHEEHLRGLITEHVQHTGSQRGEEILANWSTFATKFALVKPKSSDVKALLGHRSRSAAELRVQAQ

SEQ ID NO: 7 - protein, 472 aa, E. coli
MSQNVYQFIDLQRVDPPKKPLKIRKIEFVEIYEPFSEGQAKAQADRCLSCGNPYCEWKCPVHNYIPNWLKLANEGRIFEAAELSHQTNTLPEVCGRVCPQDRLCEGSCTLNDEFGAVTIGNIERYINDKAFEMGWRPDMSGVKQTGKKVAIIGAGPAGLACADVLTRNGVKAVVFDRHPEIGGLLTFGIPAFKLEKEVMTRRREIFTGMGIEFKLNTEVGRDVQLDDLLSDYDAVFLGVGTYQSMRGGLENEDADGVYAALPFLIANTKQLMGFGETRDEPFVSMEGKRVVVLGGGDTAMDCVRTSVRQGAKHVTCAYRRDEENMPGSRREVKNAREEGVEFKFNVQPLGIEVNGNGKVSGVKMVRTEMGEPDAKGRRRAEIVAGSEHIVPADAVIMAFGFRPHNMEWLAKHSVELDSQGRIIAPEGSDNAFQTSNPKIFAGGDIVRGSDLVVTAIAEGRKAADGIMNWLEV

SEQ ID NO: 8 - DNA, 1344 bp, Artificial Sequence
  Synthetic
atggatcagacctacagcctggagtccttcctcaatcatgtgcagaagcgcgacccgaaccaaaccgagttcgcccaggccgtgcgcgaagtcatgaccacgctctggccgttcctggaacagaaccccaagtatcggcagatgagcctgctggagcgcctggtcgaacccgagcgcgtgatccagttccgcgtggtgtgggtggacgaccgcaatcagatccaggtgaaccgggcctggcgggtccagttttcgtcggcgatcggcccgtacaagggcggcatgcgtttccacccctcggtgaacctgtccatcctgaagttcctgggcttcgagcagaccttcaagaacgcgctcaccaccctgccgatgggcggtggcaagggcggctcggacttcgaccccaagggcaagtccgaaggcgaggtgatgcgcttttgccaggccctgatgacggaactgtatcgccatctgggcgccgacaccgacgtgccggcgggcgatatcggcgtgggcggtcgcgaggtcggcttcatggccggtatgatgaagaagctgtcgaacaacacggcgtgcgtgttcaccggcaagggcctgtcgttcggcggctcgctgatccgccccgaggccacgggctacggcctggtctacttcaccgaggccatgctgaagcgccacggcatgggcttcgaaggcatgcgcgtgagcgtgtccggctccggcaacgtcgcccaatatgccatcgaaaaggcgatggagttcggtgcgcgcgtcattaccgccagcgacagctcgggcaccgtggtggacgagagcggcttcacgaaggaaaagctggcccgcctcatcgagatcaaggccagccgtgatggccgcgtcgcggactacgcgaaggagttcggcctggtgtacctcgaaggccagcaaccgtggagcctcccggtggacatcgcgctgccgtgcgccacgcagaacgaactggacgtggacgcggcccaccaactcatcgcgaacggcgtgaaggccgtcgcggaaggcgccaacatgccgaccaccatcgaggcgaccgaactgtttcaacaggccggtgtcctgttcgcgccgggcaaggccgccaatgccggcggcgtcgccacgtcgggcctggaaatggcccagaacgcggcccgcctgggctggaaggccgaaaaggtcgatgcccgcctgcaccacatcatgctggatatccatcacgcctgcgtggagcacggcggcgagggcgagcagacgaactacgtccagggtgcgaacattgcgggcttcgtcaaggtcgcggacgccatgctggcgcagggcgtgatctga

SEQ ID NO: 9 - protein, 447 aa, E. coli
MDQTYSLESFLNHVQKRDPNQTEFAQAVREVMTTLWPFLEQNPKYRQMSLLERLVEPERVIQFRVVWVDDRNQIQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKASRDGRVADYAKEFGLVYLEGQQPWSLPVDIALPCATQNELDVDAAHQLIANGVKAVAEGANMPTTIEATELFQQAGVLFAPGKAANAGGVATSGLEMAQNAARLGWKAEKVDARLHHIMLDIHHACVEHGGEGEQTNYVQGANIAGFVKVADAMLAQGVI

SEQ ID NO: 10 - DNA, 1308 bp, Artificial Sequence
  Synthetic
atgtcgagcgcggcgcccaccaacatcgccggccagaagcacgcgctgccgagctacctgaatgcggaccatctgggcccgtggggcatctatctgcagcaagtcgatcgggtgacgccctacctgggctccctggcccgctgggtcgaaaccctgaagcgcccgaagcgggccatgatcgtggacgtgccgatcgagctcgacaacggcacgatcgcccacttcgaaggctaccgtgtccagcacaacctgagccgcggtcccggcaagggcggcgtgcgtttccaccaagacgtgacgctgtccgaggtcatggccctgagcgcctggatgtcggtcaagaacgccgcggtcaatgtcccctacggcggcgccaagggcggcatccgcgtggacccgcgcacgctgtcgcacgccgagctggaacgcctcacgcggcgctacacctcggagatcaacatcatcatcggcccgtccaaggacatcccggcgccggacgtgaataccaacgcccaggtgatggcctggatgatggatacctattccatgaactcgggttcgacggcgaccggcgtggtcacgggcaagccgatcagcctcggcggctcgctgggccgtcatgaagcgacgggccgcggcgtgttcgtggtgggtagcgaggccgcgcgcaacatcggcctggagatcaagggcgcccgggtcgcggtccagggttttggcaacgtgggcgccgtggccgccaagctgttccacgaagcgggcgcgaaggtcgtcgccgtgcaggaccaccgcaccaccctgttcgatcccgcgggcctggacgtgcccgccatgatggagtacgcgtcgcatagcggcaccatcgagggctttcggggcgaggtgctgcgcaccgagcagttctgggaagtcgattgcgacatcctgattcccgccgccctggaaggccagatcaccgtgcagaacgcgccgaagattaccgccaagctcgtcatcgaaggcgcgaacggtccgacgacgccgcaggccgatgacatcctgcgcgagcgcaacatcctggtctgcccggacgtgatcgccaacgcgggcggcgtgaccgtgagctatttcgagtgggtgcaggacttctcgtcgttcttctggaccgaagaagagatcaaccagcgcctggtgcgcatcatgcaagaagcgttccgcgccatctggcaagtggcgcaggacaataaggtcaccctccgcaccgccgccttcatcgtggcctgcacccgcattctgcaggcccgcgagatgcgcggcctgtacccgtga

SEQ ID NO: 11 - protein, 435 aa, C.necator
MSSAAPTNIAGQKHALPSYLNADHLGPWGIYLQQVDRVTPYLGSLARWVETLKRPKRAMIVDVPIELDNGTIAHFEGYRVQHNLSRGPGKGGVRFHQDVTLSEVMALSAWMSVKNAAVNVPYGGAKGGIRVDPRTLSHAELERLTRRYTSEINIIIGPSKDIPAPDVNTNAQVMAWMMDTYSMNSGSTATGVVTGKPISLGGSLGRHEATGRGVFVVGSEAARNIGLEIKGARVAVQGFGNVGAVAAKLFHEAGAKVVAVQDHRTTLFDPAGLDVPAMMEYASHSGTIEGFRGEVLRTEQFWEVDCDILIPAALEGQITVQNAPKITAKLVIEGANGPTTPQADDILRERNILVCPDVIANAGGVTVSYFEWVQDFSSFFWTEEEINQRLVRIMQEAFRAIWQVAQDNKVTLRTAAFIVACTRILQAREMRGLYP

SEQ ID NO: 12 - DNA, 1071 bp, Artificial Sequence
  Synthetic
atggagtcgaaggtcgtcgtgccggcgcagggcaagaagatcacgctgcagaacggcaagctgaacgtgccggagaacccgatcatcccgtacatcgagggcgacggcatcggtgtggacgtgacgcccgccatgctgaaggtcgtggatgcggccgtggaaaaggcctacaagggcgagcgcaagatcagctggatggaaatctacaccggtgagaagtccacccaggtgtatggccaagatgtgtggctgccggccgaaacgctggacctgatccgcgagtatcgcgtcgcgatcaagggcccgctcaccaccccggtgggcggcggcatccggtcgctgaacgtcgccctgcgccaagaactggacctgtacatctgcctgcgccccgtgcgctactaccagggcaccccctcgcccgtcaagcatcccgaactcaccgacatggtgatcttccgcgaaaacagcgaggacatctacgcgggcatcgagtggaaggccgacagcgccgacgcggaaaaggtcattaagttcctgcgcgaagagatgggcgtgaagaagatccgcttcccggagcactgcggcatcggcatcaagccgtgctccgaggaaggcacgaagcgtctggtgcgtgccgccatcgaatatgcgatcgcgaacgatcgcgacagcgtcaccctggtccacaagggcaacatcatgaagtttaccgaaggcgcgttcaaggactggggctatcagctggcccgcgaagagttcggtggcgagctgatcgacggcggcccgtggctcaaggtgaagaacccgaacaccggcaaggaaattgtgattaaggatgtgatcgcggacgcgttcctgcagcagatcctgctgcggccggccgagtacgatgtcatcgccgagatgatgctgcgccacatgggctggacggaagcggccgacctgattgtgaagggcatggaaggtgccatcaatgccaagaccgtcacgtacgacttcgagcggctgatggacggcgccaagctcctcaagtgctcggagttcggcgacgccatcatcgagaatatgtga

SEQ ID NO: 13 - protein, 356 aa, E. coli
MESKVVVPAQGKKITLQNGKLNVPENPIIPYIEGDGIGVDVTPAMLKVVDAAVEKAYKGERKISWMEIYTGEKSTQVYGQDVWLPAETLDLIREYRVAIKGPLTTPVGGGIRSLNVALRQELDLYICLRPVRYYQGTPSPVKHPELTDMVIFRENSEDIYAGIEWKADSADAEKVIKFLREEMGVKKIRFPEHCGIGIKPCSEEGTKRLVRAAIEYAIANDRDSVTLVHKGNIMKFTEGAFKDWGYQLAREEFGGELIDGGPWLKVKNPNTGKEIVIKDVIADAFLQQILLRPAEYDVIAEMMLRHMGWTEAADLIVKGMEGAINAKTVTYDFERLMDGAKLLKCSEFGDAIIENM

SEQ ID NO: 14 - DNA, 2217 bp, Artificial Sequence
  Synthetic
atggcgaagatcatctggacccgcaccgacgaagcccccctcctggcgacctatagcctgaagcccgtggtggaagcgttcgcggcgacggccggcatcgaagtggaaacccgcgatatcagcctcgcgggccgcatcctggcgcagttcccggagcgcctgaccgaggatcaaaaggtcggcaacgccctggccgagctgggtgagctggccaagacccccgaggcgaacatcatcaagctgccgaacatctcggccagcgtgccgcagctcaaggccgcgattaaggaactgcaggaccagggctacgacatccccgagctgccggataatgccaccaccgacgaagaaaaggacatcctggcccgctacaacgccgtgaagggttcggcggtgaacccggtcctgcgcgaaggcaatagcgatcgccgcgccccgatcgccgtcaagaacttcgtgaagaagtttccgcaccgcatgggcgagtggtcggcggacagcaagacgaacgtggccacgatggacgccaatgacttccgccacaacgagaagtcgatcattctggatgcggccgatgaagtccagatcaagcacatcgcggccgacggcaccgaaaccatcctgaaggactcgctgaagctgctggaaggcgaagtgctggacggtaccgtgctgagcgcgaaggccctggacgccttcctgctggaacaagtggcccgcgccaaggccgagggcatcctgttctccgcccatctgaaggccaccatgatgaaggtgtccgacccgatcatcttcggccatgtcgtgcgcgcctacttcgccgatgtctttgcgcagtacggcgagcagctgctggcggccggcctgaacggtgagaacggcctggcggccattctgtccggcctggagtcgctcgacaatggcgaggaaatcaaggccgcgttcgaaaagggcctggaggacggcccggacctggcgatggtgaactcggcccgcggcatcaccaacctccacgtcccctcggacgtgatcgtggacgcctccatgccggccatgatccgcacctcgggccacatgtggaacaaggacgaccaggaacaggacacgctggccatcatccccgacagctcgtatgccggcgtctaccaaacggtcatcgaggactgccgcaagaacggcgcgttcgatccgaccacgatgggcaccgtcccgaacgtcggcctgatggcccaaaaggccgaagagtatggtagccacgacaagaccttccgcatcgaggccgacggcgtcgtccaggtcgtcagctccaatggcgacgtgctgattgaacacgacgtggaagccaacgatatctggcgcgcgtgccaggtcaaggatgcgcccatccaggactgggtgaagctggccgtgacccggtcccgcctgagcggcatgccggcggtgttctggctggacccggaacgggcccacgaccgcaacctggccagcctcgtggagaagtacctggcggaccacgataccgaaggcctggacatccagatcctgtccccggtcgaagccacgcagctgtcgatcgatcgtatccgccgtggcgaggacacgatctcggtgaccggcaacgtgctgcgcgactacaatacggacctgttcccgatcctggaactgggcaccagcgcgaagatgctgtcggtggtgcccctcatggcgggcggcggcctgttcgaaaccggcgcgggtggcagcgccccgaagcacgtgcaacaggtgcaagaagaaaaccatctgcgctgggacagcctgggcgagttcctggcgctggccgagtcgttccgccacgagctgaacaacaacggcaacacgaaggccggcgtgctcgccgacgcgctcgataaggcgaccgagaagctcctcaatgaagagaagtcgccctcgcgcaaggtcggcgagattgacaaccgcggttcccatttctggctcacgaagttttgggcggacgagctggcggcccagacggaagatgccgacctggcggcgaccttcgcccccgtcgccgaggccctcaacaccggcgcggcggacatcgacgccgcgctgctcgcggtccagggcggcgccaccgacctgggcggctactactcgccgaacgaagagaagctgaccaacatcatgcggcccgtggcgcagttcaatgaaattgtggacgccctgaagaagtga

SEQ ID NO: 15 - protein, 738 aa, C. glutamicum
MAKIIWTRTDEAPLLATYSLKPVVEAFAATAGIEVETRDISLAGRILAQFPERLTEDQKVGNALAELGELAKTPEANIIKLPNISASVPQLKAAIKELQDQGYDIPELPDNATTDEEKDILARYNAVKGSAVNPVLREGNSDRRAPIAVKNFVKKFPHRMGEWSADSKTNVATMDANDFRHNEKSIILDAADEVQIKHIAADGTETILKDSLKLLEGEVLDGTVLSAKALDAFLLEQVARAKAEGILFSAHLKATMMKVSDPIIFGHVVRAYFADVFAQYGEQLLAAGLNGENGLAAILSGLESLDNGEEIKAAFEKGLEDGPDLAMVNSARGITNLHVPSDVIVDASMPAMIRTSGHMWNKDDQEQDTLAIIPDSSYAGVYQTVIEDCRKNGAFDPTTMGTVPNVGLMAQKAEEYGSHDKTFRIEADGVVQVVSSNGDVLIEHDVEANDIWRACQVKDAPIQDWVKLAVTRSRLSGMPAVFWLDPERAHDRNLASLVEKYLADHDTEGLDIQILSPVEATQLSIDRIRRGEDTISVTGNVLRDYNTDLFPILELGTSAKMLSVVPLMAGGGLFETGAGGSAPKHVQQVQEENHLRWDSLGEFLALAESFRHELNNNGNTKAGVLADALDKATEKLLNEEKSPSRKVGEIDNRGSHFWLTKFWADELAAQTEDADLAATFAPVAEALNTGAADIDAALLAVQGGATDLGGYYSPNEEKLTNIMRPVAQFNEIVDALKK

SEQ ID NO: 16 - DNA, 1536 bp, Artificial Sequence
  Synthetic
atggccacgtcggtccagaccggtaaggccaagcaactgaccctgctgggctttttcgccatcacggcctcgatggtgatggccgtctacgagtacccgacctttgccacctcgggcttctcgctggtgttcttcctgctcctgggcggcatcctgtggttcatcccggtgggcctgtgcgcggccgagatggcgaccgtggatggctgggaagagggcggcgtgttcgcgtgggtgagcaacaccctgggcccgcgctggggctttgcggccatctccttcggctacctccagatcgcgatcggcttcatccccatgctctacttcgtcctgggtgccctgtcctatatcctcaagtggccggccctgaatgaagatccgattaccaagacgattgccgcgctgatcatcctgtgggccctggccctgacccagttcggcggcacgaagtacacggcgcgcattgcgaaggtcggcttcttcgcgggcatcctcctgccggccttcatcctgattgcgctggcggccatctatctgcactcgggcgccccggtggcgatcgagatggacagcaagaccttcttccccgacttctccaaggtcggcaccctggtcgtgttcgtcgcgttcatcctctcgtacatgggcgtcgaggccagcgccacccatgtcaacgaaatgtcgaacccgggccgcgactatcccctggcgatgctgctgctcatggtggccgccatctgcctgtcgagcgtgggcggcctgagcatcgcgatggtgatcccgggcaacgaaatcaacctgtcggcgggcgtgatgcaaacgttcaccgtgctgatgtcccacgtggccccggaaatcgagtggaccgtgcgggtgatcagcgccctgctgctgctgggtgtgctggccgagatcgcgtcctggatcgtgggcccctcgcgtggcatgtacgtgaccgcccagaagaatctgctgccggcggccttcgccaagatgaataagaacggcgtgccggtgaccctggtgatcagccagctggtcatcacctcgatcgccctcatcatcctgaccaacaccggcggcggcaacaacatgagctttctgatcgcgctggcgctgaccgtggtgatctacctgtgcgcgtacttcatgctgttcatcggttatatcgtcctggtcctgaagcatccggacctgaagcgcacgtttaacatccccggcggcaagggcgtgaagctcgtcgtggccattgtgggcctgctcacgtcgatcatggcgttcatcgtgtccttcctgcccccggacaacatccagggtgacagcacggatatgtacgtggaactgctggtcgtgtcgttcctggtggtcctggcgctgcccttcatcctctacgcggtccacgaccggaagggcaaggccaacacgggcgtcacgctggagccgatcaactcgcagaacgcccccaagggccacttcttcctccacccgcgcgcccgcagcccgcattacatcgtcatgaacgacaagaagcactga

SEQ ID NO: 17 - protein, 511 aa, E. coli
MATSVQTGKAKQLTLLGFFAITASMVMAVYEYPTFATSGFSLVFFLLLGGILWFIPVGLCAAEMATVDGWEEGGVFAWVSNTLGPRWGFAAISFGYLQIAIGFIPMLYFVLGALSYILKWPALNEDPITKTIAALIILWALALTQFGGTKYTARIAKVGFFAGILLPAFILIALAAIYLHSGAPVAIEMDSKTFFPDFSKVGTLVVFVAFILSYMGVEASATHVNEMSNPGRDYPLAMLLLMVAAICLSSVGGLSIAMVIPGNEINLSAGVMQTFTVLMSHVAPEIEWTVRVISALLLLGVLAEIASWIVGPSRGMYVTAQKNLLPAAFAKMNKNGVPVTLVISQLVITSIALIILTNTGGGNNMSFLIALALTVVIYLCAYFMLFIGYIVLVLKHPDLKRTFNIPGGKGVKLVVAIVGLLTSIMAFIVSFLPPDNIQGDSTDMYVELLVVSFLVVLALPFILYAVHDRKGKANTGVTLEPINSQNAPKGHFFLHPRARSPHYIVMNDKKH

SEQ ID NO: 18 - DNA, 28 bp, Artificial sequence
  Synthetic
gcatgggcataaagttgcctttttaatc

SEQ ID NO: 19 - DNA, 30 bp, Artificial sequence
  Synthetic
cggtgatgccacgatcctcgccctgctggc

SEQ ID NO: 20 - DNA, 29 bp, Artificial sequence
  Synthetic
ggatccgctaatcttatggataaaaatgc

SEQ ID NO: 21 - DNA, 19 bp, Artificial sequence
  Synthetic
agcattctgtaacaaagcg

SEQ ID NO: 22 - DNA, 20 bp, Artificial sequence
  Synthetic
tgcaaggcgattaagttggg

SEQ ID NO: 23 - DNA, 19 bp, Artificial sequence
  Synthetic
aagcgcgcaattaaccctc

SEQ ID NO: 24 - DNA, 20 bp, Artificial sequence
  Synthetic
gaaactctggctcaccgacg

SEQ ID NO: 25 - DNA, 20 bp, Artificial sequence
  Synthetic
gctgttcagggatttgcagc

SEQ ID NO: 26 - DNA, 50 bp, Artificial sequence
  Synthetic
atagtgagcgtcccatgatgcagcagtatctgctggacctgtaattaacg

SEQ ID NO: 27 - DNA, 50 bp, Artificial sequence
  Synthetic
gaccgttaattacaggtccagcagatactgctgcatcatgggacgctcac

SEQ ID NO: 28 - DNA, 55 bp, Artificial sequence
  Synthetic
ggtatatgtgatgggttaaaaaggatcgatgaacttgagcgcgccctcgttatcg

SEQ ID NO: 29 - DNA, 55 bp, Artificial sequence
  Synthetic
tacatcaccgacgagcaaggcaagaccgatccgccgttgctgtcatcgttttctg

SEQ ID NO: 30 - DNA, 23 bp, Artificial sequence
  Synthetic
gcaccgaggcgcgcaccggtgcg

SEQ ID NO: 31 - DNA, 21 bp, Artificial sequence
  Synthetic
gaacgcgggcagcgcttccag

SEQ ID NO: 32 - DNA, 20 bp, Artificial sequence
  Synthetic
atcggccaccatgttgacgc

SEQ ID NO: 33 - DNA, 20 bp, Artificial sequence
  Synthetic
tctgcttcaagctgaaggac

SEQ ID NO: 34 - DNA, 21 bp, Artificial sequence
  Synthetic
atgttcttgtcgaaggactcg

SEQ ID NO: 35 - DNA, 20 bp, Artificial sequence
  Synthetic
cggtgctcgcctggaagctg

SEQ ID NO: 36 - DNA, 20 bp, Artificial sequence
  Synthetic
gcccacggcgtagttcttcg

SEQ ID NO: 37 - DNA, 20 bp, Artificial sequence
  Synthetic
tggccttcggcttccgtccg

SEQ ID NO: 38 - DNA, 20 bp, Artificial sequence
  Synthetic
tcaccgaggggtggaaacgc

SEQ ID NO: 39 - DNA, 20 bp, Artificial sequence
  Synthetic
cgggcaaggccgccaatgcc

SEQ ID NO: 40 - DNA, 20 bp, Artificial sequence
  Synthetic
tgggcgatcgtgccgttgtc

SEQ ID NO: 41 - DNA, 20 bp, Artificial sequence
  Synthetic
ccggacgtgatcgccaacgc

SEQ ID NO: 42 - DNA, 20 bp, Artificial sequence
  Synthetic
cacctgggtggacttctcac

SEQ ID NO: 43 - DNA, 20 bp, Artificial sequence
  Synthetic
atcagctggcccgcgaagag

SEQ ID NO: 44 - DNA, 20 bp, Artificial sequence
  Synthetic
tggcattatccggcagctcg

SEQ ID NO: 45 - DNA, 20 bp, Artificial sequence
  Synthetic
cctcaatgaagagaagtcgc

SEQ ID NO: 46 - DNA, 35 bp, Artificial sequence
  Synthetic
atgtacggtctcaggataaaggaggtatatcgatg

SEQ ID NO: 47 - DNA, 35 bp, Artificial sequence
  Synthetic
atgtacggtctcatatgaaaggaggtatatcgatg

SEQ ID NO: 48 - DNA, 35 bp, Artificial sequence
  Synthetic
atgtacggtctcaatcgaaaggaggtatatcgatg

SEQ ID NO: 49 - DNA, 35 bp, Artificial sequence
  Synthetic
atgatcggtctctagtacaacgtaggaagagtttg

SEQ ID NO: 50 - DNA, 23 bp, Artificial sequence
  Synthetic
cgctctcctgagtaggacaaatc

SEQ ID NO: 51 - DNA, 36 bp, Artificial sequence
  Synthetic
atgatcggtctctatcctcacacttccagccaattc

SEQ ID NO: 52 - DNA, 35 bp, Artificial sequence
  Synthetic
atgatcggtctctatcctcagatcacgccctgcgc

SEQ ID NO: 53 - DNA, 34 bp, Artificial sequence
  Synthetic
atgatcggtctctatcctcacgggtacaggccgc

SEQ ID NO: 54 - DNA, 36 bp, Artificial sequence
  Synthetic
atgatcggtctctgccatcacttcaggacgtagccg

SEQ ID NO: 55 - DNA, 19 bp, Artificial sequence
  Synthetic
gaagggcaaggccaacacg

SEQ ID NO: 56 - DNA, 19 bp, Artificial sequence
  Synthetic
gtactcgtagacggccatc

SEQ ID NO: 57 - DNA, 20 bp, Artificial sequence
  Synthetic
gtggtgccgggcgaggtgtg

SEQ ID NO: 58 - DNA, 20 bp, Artificial sequence
  Synthetic
gaacaccggcagatggaaac

SEQ ID NO: 59 - DNA, 20 bp, Artificial sequence
  Synthetic
agagcttcaaggaccgtctg

SEQ ID NO: 60 - DNA, 20 bp, Artificial sequence
  Synthetic
catgcccttgatcacatcgc

SEQ ID NO: 61 - DNA, 19 bp, Artificial sequence
  Synthetic
gtgggcggcatcagctacc

SEQ ID NO: 62 - DNA, 21 bp, Artificial sequence
  Synthetic
gatgtcgcccaggtcctcgac

SEQ ID NO: 63 - DNA, 20 bp, Artificial sequence
  Synthetic
tgatcatcgccctggtctgc

SEQ ID NO: 64 - DNA, 20 bp, Artificial sequence
  Synthetic
aggggcagggccgtatacag

SEQ ID NO: 65 - DNA, 20 bp, Artificial sequence
  Synthetic
cttcccgggcttcgagcacg

SEQ ID NO: 66 - DNA, 20 bp, Artificial sequence
  Synthetic
caggcgctccacggcgttac

SEQ ID NO: 67 - DNA, 20 bp, Artificial sequence
  Synthetic
tgaaggacctgaacatcgtc

SEQ ID NO: 68 - DNA, 19 bp, Artificial sequence
  Synthetic
catgggcaggcgcaggaag

SEQ ID NO: 69 - DNA, 19 bp, Artificial sequence
  Synthetic
caagaccggtgatggctgc

SEQ ID NO: 70 - DNA, 20 bp, Artificial sequence
  Synthetic
atcaccggcaaccgccagtg

SEQ ID NO: 71 - DNA, 20 bp, Artificial sequence
  Synthetic
gaagaggtgggctcgcgcga

SEQ ID NO: 72 - DNA, 20 bp, Artificial sequence
  Synthetic
accagtcgctgcgctgcgac

SEQ ID NO: 73 - DNA, 18 bp, Artificial sequence
  Synthetic
gaacgccgtgaacatcgc

SEQ ID NO: 74 - DNA, 19 bp, Artificial sequence
  Synthetic
ctgggagctgggcctcgtg

SEQ ID NO: 75 - DNA, 20 bp, Artificial sequence
  Synthetic
ctccctgtcgggctatatcg

SEQ ID NO: 76 - DNA, 19 bp, Artificial sequence
  Synthetic
ctggagcaccttcgcgacg

SEQ ID NO: 77 - DNA, 21 bp, Artificial sequence
  Synthetic
gcaagaaggtcgcgatcatcg

SEQ ID NO: 78 - DNA, 19 bp, Artificial sequence
  Synthetic
cgtgatccagttccgcgtg

SEQ ID NO: 79 - DNA, 19 bp, Artificial sequence
  Synthetic
cggtgcgcgcgtcattacc

SEQ ID NO: 80 - DNA, 19 bp, Artificial sequence
  Synthetic
caagtcgatcgggtgacgc

SEQ ID NO: 81 - DNA, 19 bp, Artificial sequence
  Synthetic
ccagggttttggcaacgtg

SEQ ID NO: 82 - DNA, 20 bp, Artificial sequence
  Synthetic
gtaaggccaagcaactgacc

SEQ ID NO: 83 - DNA, 20 bp, Artificial sequence
  Synthetic
cgttcatcctctcgtacatg

SEQ ID NO: 84 - DNA, 20 bp, Artificial sequence
  Synthetic
cggcggcaagggcgtgaagc

SEQ ID NO: 85 - DNA, 19 bp, Artificial sequence
  Synthetic
ggccgcggtggtcatcacg

SEQ ID NO: 86 - DNA, 19 bp, Artificial sequence
  Synthetic
cggtctacctggaagcgag

SEQ ID NO: 87 - DNA, 20 bp, Artificial sequence
  Synthetic
gctgtttcggcccttcatcc

SEQ ID NO: 88 - DNA, 20 bp, Artificial sequence
  Synthetic
cccaccaagacgcagcgctg

SEQ ID NO: 89 - DNA, 20 bp, Artificial sequence
  Synthetic
tccttctatgccctgctcac

SEQ ID NO: 90 - DNA, 19 bp, Artificial sequence
  Synthetic
cgccggcctgttcttcatg

SEQ ID NO: 91 - DNA, 40 bp, Artificial sequence
  Synthetic
atgtacggtctcagtatctgctggacctgtaattaacggt

SEQ ID NO: 92 - DNA, 33 bp, Artificial sequence
  Synthetic
gtacatggtctctatactgctgcatcatgggac

SEQ ID NO: 93 - DNA, 35 bp, Artificial sequence
  Synthetic
gtacatggtctctagtagaacttgagcgcgccctc

SEQ ID NO: 94 - DNA, 31 bp, Artificial sequence
  Synthetic
atgtacggtctcaatcgccgccgttgctgtc

SEQ ID NO: 95 - DNA, 38 bp, Artificial sequence
  Synthetic
atgtacggtctcatatgaaaggaggtatatcgatgctg

SEQ ID NO: 96 - DNA, 20 bp, Artificial sequence
  Synthetic
ctggacgcgcagtcgctgtg

SEQ ID NO: 97 - DNA, 20 bp, Artificial sequence
  Synthetic
gatatcccggccgttcgagc

SEQ ID NO: 98 - DNA, 20 bp, Artificial sequence
  Synthetic
tgggcctggtggccatgaag

(19) United States
(12) Patent Application Publication
(10) Pub. No.: US 2019/0233860 A1
(43) Pub. Date: Aug. 1, 2019

(54) METHODS AND MATERIALS FOR THE BIOSYNTHESIS OF COMPOUNDS INVOLVED IN GLUTAMATE METABOLISM AND DERIVATIVES AND COMPOUNDS RELATED THERETO

(71) Applicant: INVISTA NORTH AMERICA S.A.R.L., Wilmington, DE (US)

(72) Inventors: Katherine Louise Tibbles, Redcar (GB); Alexander Brett Foster, Redcar (GB)

(73) Assignee: INVISTA NORTH AMERICA S.A.R.L., Wilmington, DE (US)

(21) Appl. No.: 16/264,751

(22) Filed: Feb. 1, 2019

Related U.S. Application Data
(60) Provisional application No. 62/624,895, filed on Feb. 1, 2018.

(51) Int. Cl. C12P 13/14; C12N 9/88; C12N 9/04; C12N 9/06

(52) U.S. Cl. CPC C12P 13/14; C12N 9/88; C12Y 401/01015; C12Y 104/01013; C12Y 101/01042; C12N 9/0016; C12N 9/0006

(57) ABSTRACT

Methods and materials for the biosynthesis of compounds involved in glutamate metabolism, and derivatives and compounds related thereto are provided. Also provided are products produced in accordance with these methods and materials.

This patent application claims the benefit of priority from U.S. Provisional Application Ser. No. 62/624,895 filed Feb. 1, 2018, the contents of which is herein incorporated by reference in its entirety.

### FIELD

The present invention relates to biosynthetic methods and materials for the production of compounds involved in glutamate metabolism, and/or derivatives thereof and/or other compounds related thereto. The present invention also relates to products biosynthesized or otherwise encompassed by these methods and materials.

Replacement of traditional chemical production processes relying on, for example fossil fuels and/or potentially toxic chemicals, with environmentally friendly (e.g., green chemicals) and/or “cleantech” solutions is being considered, including work to identify building blocks suitable for use in the manufacturing of such chemicals. See, “Conservative evolution and industrial metabolism in Green Chemistry”, Green Chem., 2018, 20, 2171-2191.

Biosynthetic routes have been examined for production of compounds involved in glutamate metabolism. Glutamate metabolism plays a vital role in biosynthesis of nucleic acids and proteins and has been reported to be involved in various biological responses, such as different stress responses (Yelamanchi et al. J. Cell. Commun. Signal 2015 10(1):69-75).

One option for biosynthesis has been reported to be the synthesis of γ-aminobutyrate (GABA) directly from glutamate. To synthesize GABA in vivo, carbon is diverted out of the TCA cycle by conversion of α-ketoglutarate to glutamate. Glutamate is then decarboxylated by glutamate decarboxylase (GDC) to form GABA. E. coli strains producing GABA using GDC have been described by le Vo et al. (Bioprocess and biosystems engineering 2012 35(4):645-650), Lee et al. (Journal of Biotechnology 2015 207:52-57) and Somasundaram et al. (Journal of industrial microbiology & biotechnology 2016 43(1): 79-86). Production of GABA from glucose in C. glutamicum has also been disclosed by Shi et al. (Biotechnology letters 2011 33(12): 2469-2474) and Wang et al. (Biotechnology letters 2015 37(7):1473-1481). However, difficulty has been observed, for example involving overexpressing genes to bioengineer carbon flux to glutamate (as described by Lee et al. (Journal of Biotechnology 2015 207:52-57)), for the native TCA cycle genes isocitrate dehydrogenase and glutamate synthase in E. coli, which was not successful in C. glutamicum. Instead, attenuation or deletion of a competing enzyme was reported as a viable approach in C. glutamicum (Eikmanns et al. Journal of bacteriology 1995 177(3):774-782; Asakura et al. Applied and environmental microbiology 2007 73(4):1308-1319).

Bacteria that use GABA production to tolerate low pH have a GABA/glutamate antiporter, which reportedly allows the hosts to import extracellular glutamate and export GABA (Small & Waterman Trends in microbiology 1998 6(6):214-216). In E. coli, co-overexpression of a GDC and a GABA antiporter (GadC; P63235) increased GABA production by up to 38% vs GDC alone, and scaffolding of GDC to the GABA antiporter increased GABA yields further (le Vo et al. Bioprocess and biosystems engineering 2012 35(4):645-650; Somasundaram et al. Journal of industrial microbiology & biotechnology 2016 43(1): 79-86). The GABA antiporter is most active at low pH (<6) and requires glutamate in the media.

Biosynthetic materials and methods, including organisms having increased production of compounds involved in glutamate metabolism, derivatives thereof and compounds related thereto are needed.

### SUMMARY OF THE INVENTION

An aspect of the present invention relates to a process for the biosynthesis of compounds involved in glutamate metabolism and/or derivative compounds and/or compounds related thereto. The present invention includes a process comprising obtaining an organism capable of producing compounds involved in glutamate metabolism and derivatives and compounds related thereto, altering the organism, and producing more compounds involved in glutamate metabolism and derivatives and compounds related thereto in the altered organism as compared to the unaltered organism. In one nonlimiting embodiment, the organism is C. necator or an organism with properties similar thereto.

In one nonlimiting embodiment, the organism is altered to express a glutamate decarboxylase (GDC).

In one nonlimiting embodiment, the organism is altered to express or overexpress enzymes such as, but not limited to, isocitrate dehydrogenase, glutamate dehydrogenase and glutamate synthase.

In one nonlimiting embodiment, the organism is altered to express a GABA antiporter.

In one nonlimiting embodiment, the organism is altered by deleting one or more genes which encode enzymes which degrade GABA such as, but not limited to an aminobutyrate aminotransferase (gabT) gene.

In one nonlimiting embodiment, the organism is altered by redirecting carbon towards glutamate and deleting competing pathways. In one nonlimiting embodiment, genes encoding 2-ketoglutarate dehydrogenase, and subunits thereof are deleted (e.g., odhA and odhB).

In one nonlimiting embodiment, the inserted nucleic acid sequence is codon optimized for C. necator.

In one nonlimiting embodiment, the organism is further modified to eliminate phaCAB, involved in PHBs production and/or H16-A0006-9 encoding endonucleases thereby improving transformation efficiency.

Another aspect of the present invention relates to an organism altered to produce more compounds involved in glutamate metabolism and/or derivatives and compounds related thereto as compared to the unaltered organism. In one nonlimiting embodiment, the organism is C. necator or an organism with properties similar thereto.

In one nonlimiting embodiment, the organism is altered to express a GDC.

In one nonlimiting embodiment, the organism is altered to express or overexpress an enzyme in the TCA cycle such as, but not limited to, isocitrate dehydrogenase, glutamate dehydrogenase and glutamate synthase.

In one nonlimiting embodiment, the organism is altered to express a GABA antiporter.

In one nonlimiting embodiment, the organism is altered by deleting one or more genes which degrade GABA such as, but not limited to a gabT gene.

In one nonlimiting embodiment, the organism is altered by redirecting carbon towards glutamate and deleting competing pathways. In one nonlimiting embodiment, OdhA and subunits thereof such as OdhB are deleted.

In one nonlimiting embodiment, the organism is altered with a nucleic acid sequence codon optimized for C. necator.

In one nonlimiting embodiment, the organism is further modified to eliminate phaCAB, involved in PHBs production and/or H16-A0006-9 encoding endonucleases thereby improving transformation efficiency.

Another aspect of the present invention relates to bio-derived, bio-based, or fermentation-derived products produced from any of the methods and/or altered organisms disclosed herein. Such products include compositions comprising at least one bio-derived, bio-based, or fermentation-derived compound or any combination thereof, as well as bio-derived, bio-based, or fermentation-derived dietary supplements comprising these bio-derived, bio-based, or fermentation-derived compositions or compounds; formulations of bio-derived, bio-based, or fermentation-derived compositions or compounds or dietary supplements or combinations thereof; molded substances obtained by molding the bio-derived, bio-based, or fermentation-derived compositions or compounds; and bio-derived, bio-based, or fermentation-derived semi-solids or non-semi-solid streams comprising the bio-derived, bio-based, or fermentation-derived compositions or compounds, dietary supplements, molded substances or formulations, or any combination thereof.

Another aspect of the present invention relates to a bio-derived, bio-based or fermentation derived product biosynthesized in accordance with the exemplary central metabolism depicted in FIG. 1.

Another aspect of the present invention relates to exogenous genetic molecules of the altered organisms disclosed herein. In one nonlimiting embodiment, the exogenous genetic molecule comprises a codon optimized nucleic acid sequence encoding a GDC. In one nonlimiting embodiment, the exogenous genetic molecule comprises a codon optimized nucleic acid sequence encoding an enzyme such as, but not limited to, isocitrate dehydrogenase, glutamate dehydrogenase and glutamate synthase. In one nonlimiting embodiment, the exogenous genetic molecule comprises a codon optimized nucleic acid sequence encoding a GABA antiporter. Additional nonlimiting examples of exogenous genetic molecules include expression constructs and synthetic operons of one or more of GDC, isocitrate dehydrogenase, glutamate dehydrogenase glutamate synthase and/or GABA antiporter. Additional nonlimiting examples comprise altered organisms having one or more changes associated with reactants, products or reactions depicted in FIG. 1.

Another aspect of the present invention relates to means and processes for use of these means for biosynthesis of compounds involved in glutamate metabolism, and derivative compounds and compounds related thereto.

Yet another aspect of the present invention relates to synthetic molecular probes.

### BRIEF DESCRIPTION OF THE FIGURES

FIG. 1 provides a summary of pathways for production of compounds involved in glutamate metabolism, including the TCA cycle, glyoxylate bypass and GABA degradation reaction. The dashed arrow indicates the GDC reaction, not known to be native to Cupriavidus necator. The reactions in the pathway of the present invention are highlighted with EC numbers in a box or circle. The reaction catalyzed by GabT is indicated by an “X”, as the gene encoding this enzyme is to be inactivated.

FIGS. 2A and 2B show GABA (FIG. 2A) and glutamate (FIG. 2B) production in supernatant (ppm/OD) for different strains and constructs in assay 1, as depicted. The base strain was ΔphaCABΔA0006-9 and the GabTdel strain was ΔphaCABΔA0006-9ΔgabT. Construct labels (e.g., “A1”) are described in Table 2.

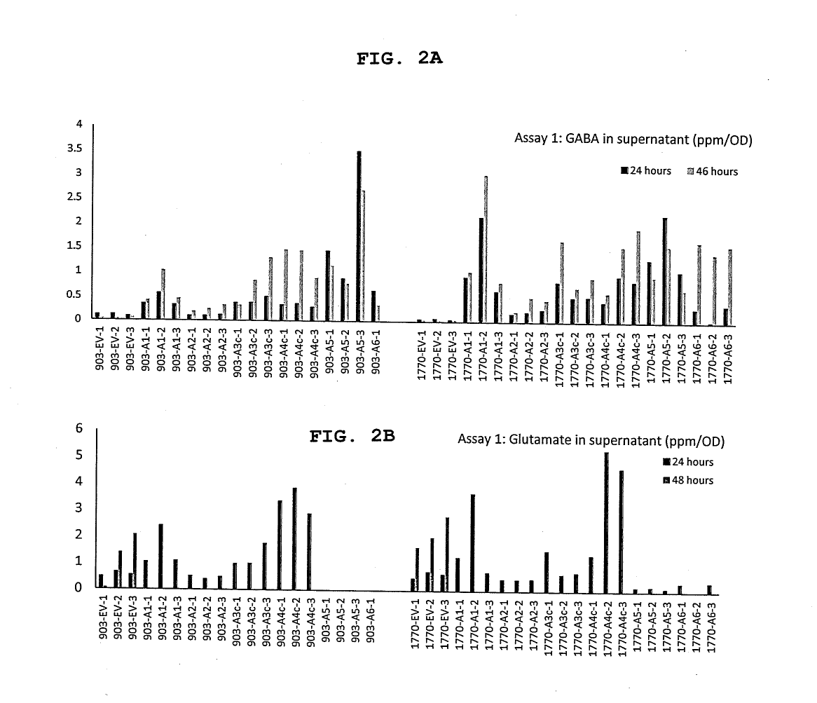

FIG. 3 shows glutamate production in supernatant (ppm/OD) for different strains and constructs according to assay 1. The base strain was ΔphaCΔBAA0006-9 and the GabTdel strain was ΔphaCABΔA0006-9ΔgabT. Construct labels are described in Table 2.

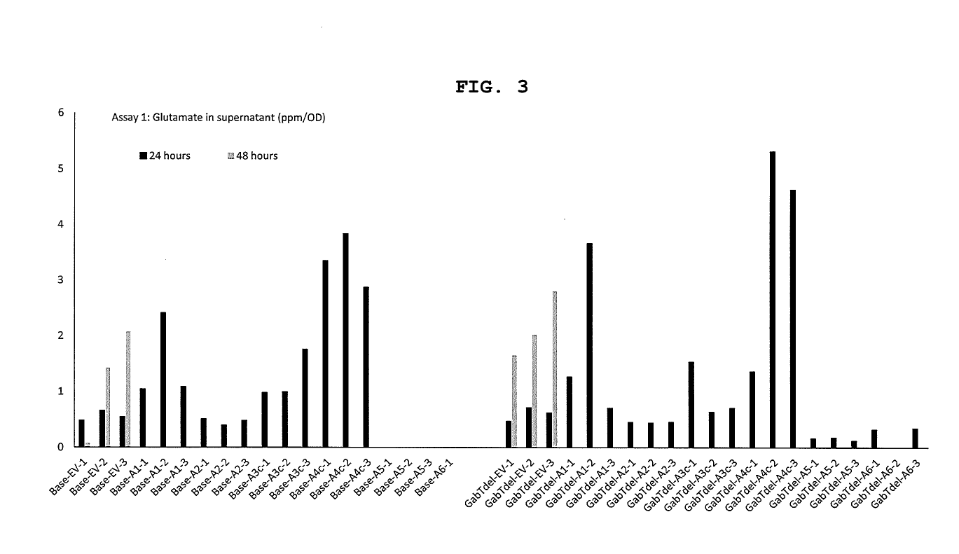

FIG. 4 shows GABA production in supernatant (ppm/OD) for different expression constructs according to assay 3. All strains were base strain (ΔphaCABΔA0006-9) and expressed E. coli GDC. Construct labels are described in Table 2.

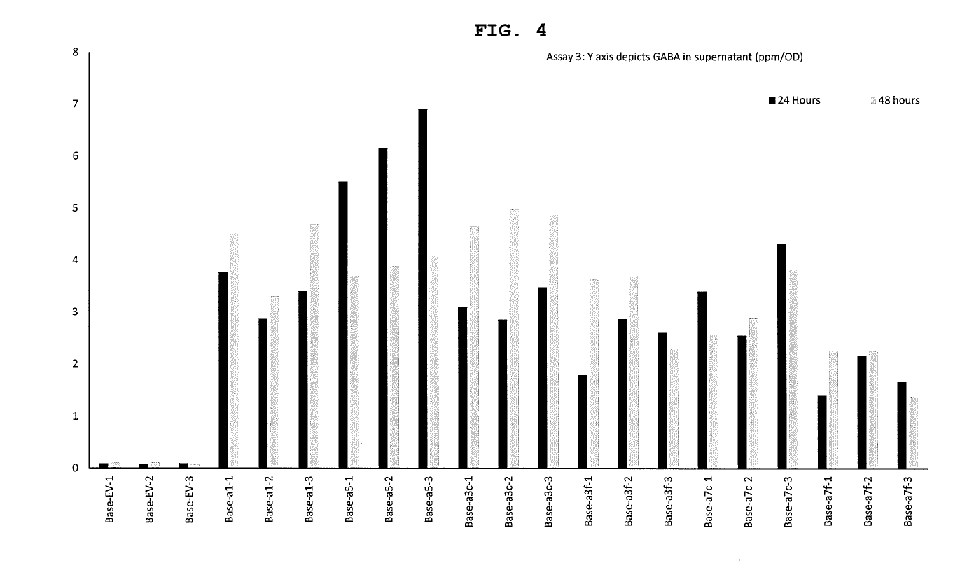

FIG. 5 shows GABA production in an Ambr15f assay in supernatant (ppm) for different strains and expression constructs. The base strain was ΔphaCABΔA0006-9 and the GabTdel strain was ΔphaCABΔA0006-9ΔgabT. Construct labels are described in Table 2.

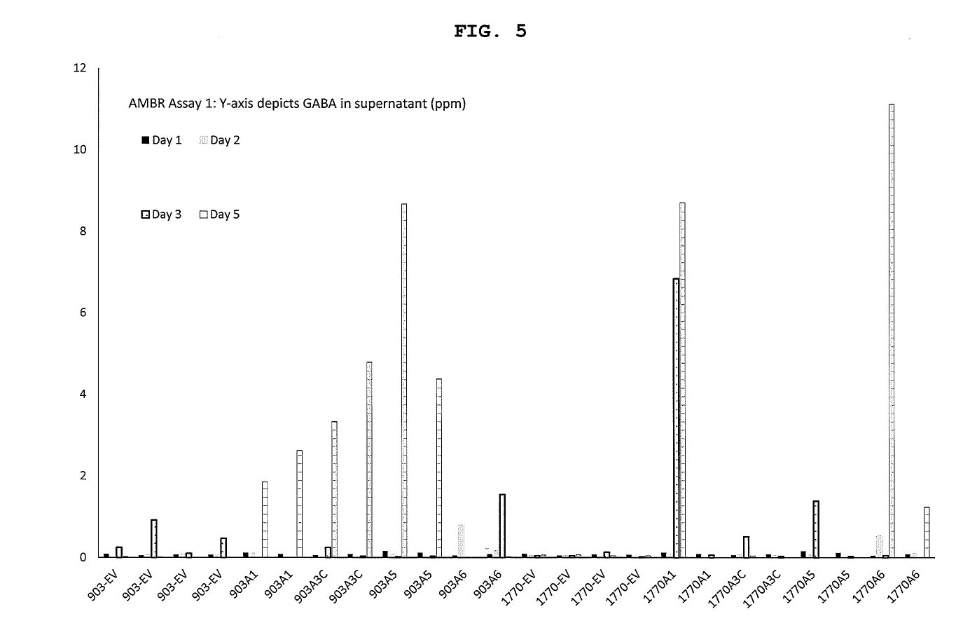

### DETAILED DESCRIPTION

The present invention provides processes for the biosynthesis of compounds involved in glutamate metabolism, and/or derivatives thereof and/or compounds related thereto as well as organisms altered to increase biosynthesis of compounds involved in glutamate metabolism, derivatives thereof and compounds related thereto, exogenous genetic molecules of these altered organisms, and bio-derived, bio-based, or fermentation-derived products biosynthesized or otherwise produced by any of these methods and/or altered organisms.

In the present invention, an organism is engineered, or redirected, to produce compounds involved in glutamate metabolism, as well as derivatives and compounds related thereto by alteration of one or more of the following nonlimiting exemplary aspects, including polypeptides having the activity of one or more of the following molecules.

In one nonlimiting embodiment, the organism is altered to express a GDC.

In one nonlimiting embodiment, the organism is altered to express or overexpress one or more such as, but not limited to, isocitrate dehydrogenase, glutamate dehydrogenase and glutamate synthase.

In one nonlimiting embodiment, the organism is altered to express a GABA antiporter.

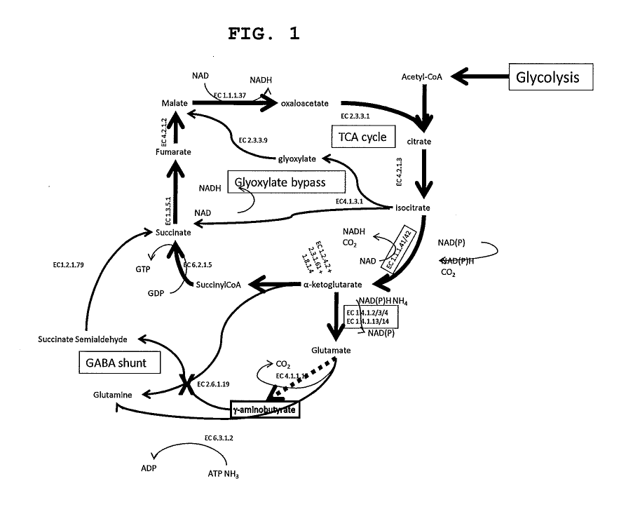

In one nonlimiting embodiment, the organism is altered by deleting one or more genes which degrade GABA such as, but not limited to a gabT gene. In another nonlimiting embodiment, the organism is altered to express, overexpress, not express or express less of one or more molecules depicted in FIG. 1. In one nonlimiting embodiment, the molecule(s) comprise a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence corresponding to a molecule(s) depicted in FIG. 1, or a functional fragment thereof.

In one nonlimiting embodiment, the organism is altered by redirecting carbon to form glutamate and deleting competing pathways (e.g., biochemical pathways that directly or indirectly utilize carbon to form compounds other than glutamate). In one nonlimiting embodiment, OdhA and subunits thereof such as OdhB are deleted.

Organisms produced in accordance with the present invention, with one or more of the above-described alterations, are expected to be useful in methods for biosynthesizing higher levels of compounds involved in glutamate metabolism, derivatives thereof, and compounds related thereto.

For purposes of the present disclosure “compounds involved in glutamate metabolism” include γ-amino butyric acid (GABA), arginine, glutamic acid, ornithine, putrescine and other C4 compounds and C5 amino acid derivative compounds.

For purposes of the present disclosure “derivatives and/or compounds related thereto” include compounds derived from the same substrates and/or enzymatic reactions as compounds involved in glutamate metabolism, byproducts of these enzymatic reactions and compounds with similar chemical structure(s) including, but not limited to, structural analogs wherein one or more substituents of compounds involved in glutamate metabolism are replaced with alternative substituents. For example, other C4 compounds and C5 amino acid derivatives include, but are not limited to 1-ornithine, butanedioic acid, 1,4-butanediol, butanoic acid, 2-amino-pentanedioic acid, and 2-pyrrolidinone. As will be understood by the skilled artisan, however, this list is exemplary only and in no way exhaustive.

For purposes of the present invention, by “higher levels of compounds involved in glutamate metabolism” it is meant that the altered organisms and methods of the present invention are capable of producing increased levels of compounds involved in glutamate metabolism and derivatives and compounds related thereto as compared to the same organism without alteration. In one nonlimiting embodiment, levels are increased by 2-fold or higher.

For compounds containing carboxylic acid groups such as organic monoacids, hydroxyacids, amino acids and dicarboxylic acids, these compounds may be formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate and/or bicarbonate, sodium hydroxide, ammonia and the like. The salt can be isolated as is from the system as the salt or converted to the free acid by reducing the pH to, for example, below the lowest pKa through addition of acid or treatment with an acidic ion exchange resin.

For compounds containing amine groups such as, but not limited to, organic amines, amino acids and diamine, these compounds may be formed or converted to their ionic salt form by addition of an acidic proton to the amine to form the ammonium salt, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid or muconic acid, and the like. The salt can be isolated as is from the system as a salt or converted to the free amine by raising the pH to, for example, above the highest pKa through addition of base or treatment with a basic ion exchange resin. Acceptable inorganic bases are known in the art and include aluminum hydroxide, calcium hydroxide, potassium hydroxide, sodium carbonate or bicarbonate, sodium hydroxide, and the like.

For compounds containing both amine groups and carboxylic acid groups such as, but not limited to, amino acids, these compounds may be formed or converted to their ionic salt form by either 1) acid addition salts, formed with inorganic acids such as hydrochloric acid, hydrobromic acid, sulfuric acid, nitric acid, phosphoric acid, and the like; or formed with organic acids such as carbonic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include aluminum hydroxide, calcium hydroxide, potassium hydroxide, sodium carbonate and/or bicarbonate, sodium hydroxide, and the like, or 2) when an acidic proton present in the parent compound either is replaced by a metal ion, e.g., an alkali metal ion, an alkaline earth ion, or an aluminum ion; or coordinates with an organic base. Acceptable organic bases are known in the art and include ethanolamine, diethanolamine, triethanolamine, trimethylamine, N-methylglucamine, and the like. Acceptable inorganic bases are known in the art and include aluminum hydroxide, calcium hydroxide, potassium hydroxide, sodium carbonate, sodium hydroxide, ammonia and the like. The salt can be isolated as is from the system or converted to the free acid by reducing the pH to, for example, below the pKa through addition of acid or treatment with an acidic ion exchange resin. In one or more aspects of the invention, it is understood that the amino acid salt can be isolated as: i. at low pH, as the ammonium (salt)-free acid form; ii. at high pH, as the amine-carboxylic acid salt form; and/or iii. at neutral or midrange pH, as the free-amine acid form or zwitterion form.

In the processes for the biosynthesis of compounds involved in glutamate metabolism and derivatives and compounds related thereto of the present invention, an organism capable of producing compounds involved in glutamate metabolism and derivatives and compounds related thereto is obtained. The organism is altered to produce more compounds involved in glutamate metabolism and derivatives and compounds related thereto in the altered organism, as compared to the unaltered organism.

In one nonlimiting embodiment, the organism is Cupriavidus necator (C. necator) or an organism having one or more properties similar thereto. A nonlimiting embodiment of the organism is set for at lgcstandards-atcc with the extension.org/products/all/17699.aspx?geo_country=gb#generalinformation of the world wide web.

C. necator (previously called Hydrogenomonas eutrophus, Alcaligenes eutropha, Ralstonia eutropha, and Wautersia eutropha) is a Gram-negative, flagellated soil bacterium of the Betaproteobacteria class. This hydrogen-oxidizing bacterium is capable of growing at the interface of anaerobic and aerobic environments and easily adapts between heterotrophic and autotrophic lifestyles. Sources of energy for the bacterium include both organic compounds and hydrogen. C. necator does not naturally contain genes for GDC and therefore does not express this enzyme. Additional properties of C. necator include microaerophilicity, copper resistance (Makar, N. S. & Casida, L. E. Int. J. of Systematic Bacteriology 1987 37(4): 323-326), bacterial predation (Byrd et al. Can J Microbiol 1985 31:1157-1163; Sillman, C. E. & Casida, L. E. Can J Microbiol 1986 32:760-762; Zeph, L. E. & Casida, L. E. Applied and Environmental Microbiology 1986 52(4):819-823) and polyhydroxybutyrate (PHB) synthesis. In addition, the cells have been reported to be capable of both aerobic and nitrate dependent anaerobic growth. A nonlimiting example of a C. necator organism useful in the present invention is a C. necator of the H16 strain. In one nonlimiting embodiment, a C. necator host of the H16 strain with at least a portion of the phaCAB gene locus knocked out (ΔphaCAB) is used.

In another nonlimiting embodiment, the organism altered in the process of the present invention has one or more of the above-mentioned properties of Cupriavidus necator.

In another nonlimiting embodiment, the organism is selected from members of the genera Ralstonia, Wautersia, Cupriavidus, Alcaligenes, Burkholderia or Pandoraea.

For the process of the present invention, the organism is engineered or redirected to produce compounds involved in glutamate metabolism, as well as derivatives and compounds related thereto by alteration of one or more of the following.

In one nonlimiting embodiment, the organism is altered to express a GDC. In one nonlimiting embodiment, the GDC is from E. coli or B. megaterium. In one nonlimiting embodiment, the GDC comprises SEQ ID NO:2 or 4 or a polypeptide with similar enzymatic activities exhibiting at least about 50%, 60%, 70%, 75%, 80%, 85%, 90%, 910, 92%, 93%, 94%, 95%, 960, 97%, 98%, 99% or 99.5% sequence identity to an amino acid sequence set forth in SEQ ID NO:2 or 4 or a functional fragment thereof. In one nonlimiting embodiment, the GDC is encoded by a nucleic acid sequence comprising SEQ ID NO:1 or 3 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 or a functional fragment thereof.

In one nonlimiting embodiment, the organism is altered to express or overexpress one or more enzymes such as, but not limited to, isocitrate dehydrogenase, glutamate dehydrogenase and glutamate synthase.

In one nonlimiting embodiment, the isocitrate dehydrogenase is from E. coli or C. glutamicum. In one nonlimiting embodiment, the isocitrate dehydrogenase comprises SEQ ID NO:13 or 15 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:13 or 15 or a functional fragment thereof. In one nonlimiting embodiment, the isocitrate dehydrogenase is encoded by a nucleic acid sequence comprising SEQ ID NO:12 or 14 or a nucleic acid sequence encoding a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to the nucleic acid sequence set forth in SEQ ID NO:12 or 14 or a functional fragment thereof.

In one nonlimiting embodiment, the glutamate dehydrogenase is from E. coli or C. necator. In one nonlimiting embodiment, the glutamate dehydrogenase comprises SEQ ID NO:9 or 11 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:9 or 11 or a functional fragment thereof. In one nonlimiting embodiment, the glutamate dehydrogenase is encoded by a nucleic acid sequence comprising SEQ ID NO:8 or 10 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 8 or 10 or a functional fragment thereof.

In one nonlimiting embodiment, the glutamate synthase is from E. coli. In one nonlimiting embodiment, the glutamate synthase comprises SEQ ID NO:6 or 7 or a polypeptide with similar enzymatic activities exhibiting at least about 50%, 60%, 70%, 75%, 80%, 850, 90%, 91%, 920, 93%, 94%, 95%, 96%, 97%, 98%, 99% or 99.5% sequence identity to an amino acid sequence set forth in SEQ ID NO:6 or 7 or a functional fragment thereof. In one nonlimiting embodiment, the glutamate synthase is encoded by a nucleic acid sequence comprising SEQ ID NO:5 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:5 or a functional fragment thereof.

In one nonlimiting embodiment, the organism is altered to express a GABA antiporter. In one nonlimiting embodiment, the GABA antiporter is from E. coli. In one nonlimiting embodiment, the GABA antiporter comprises SEQ ID NO:17 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:17 or a functional fragment thereof. In one nonlimiting embodiment, the GABA antiporter is encoded by a nucleic acid sequence comprising SEQ ID NO:16 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:16 or a functional fragment thereof.

In one nonlimiting embodiment, the organism is altered by deleting one or more genes which encode enzymes which degrade GABA such as, but not limited to a gabT gene.

In one nonlimiting embodiment, the organism is altered by redirecting carbon towards glutamate and deleting competing pathways. In one nonlimiting embodiment, OdhA and subunits thereof such as OdhB are deleted.

Organisms produced in accordance with the present invention may comprise one, two, three, four or all five of the above-described alterations,

In one nonlimiting embodiment, the nucleic acid sequence is codon optimized for C. necator.

In one nonlimiting embodiment, the organism is further modified to eliminate phaCAB, involved in PHBs production and/or H16-A0006-9 encoding endonucleases thereby improving transformation efficiency as described in U.S. patent application Ser. No. 15/717,216, teachings of which are incorporated herein by reference.

In the process of the present invention, the altered organism is then subjected to conditions wherein compounds involved in glutamate metabolism and derivatives and compounds related thereto are produced.

In the process described herein, in one aspect a fermentation strategy can be used that entails anaerobic, micro-aerobic or aerobic cultivation. In one aspect, the fermentation strategy can entail nutrient limitation such as nitrogen, phosphate or oxygen limitation, or any combination thereof.

Under conditions of nutrient limitation a phenomenon known as overflow metabolism (also known as energy spilling, uncoupling or spillage) occurs in many bacteria (Russell, 2007). In growth conditions in which there is a relative excess of carbon source and other nutrients (e.g. phosphorous, nitrogen and/or oxygen) are limiting cell growth, overflow metabolism results in the use of this excess energy (or carbon), not for biomass formation but for the excretion of metabolites, typically organic acids.

In Cupriavidus necator a modified form of overflow metabolism occurs in which excess carbon is utilized or “sunk” intracellularly, into the storage carbohydrate polyhydroxybutyrate (PHB). In strains of C. necator which are deficient in PHB synthesis this overflow metabolism can result in the production of extracellular overflow metabolites. The range of metabolites that have been detected in PHB deficient C. necator strains include acetate, acetone, butanoate, cis-aconitate, citrate, ethanol, fumarate, 3-hydroxybutanoate, propan-2-ol, malate, methanol, 2-methyl-propanoate, 2-methyl-butanoate, 3-methyl-butanoate, 2-oxoglutarate, meso-2,3-butanediol, acetoin, DL-2,3-butanediol, 2-methylpropan-1-ol, propan-1-ol, lactate 2-oxo-3-methylbutanoate, 2-oxo-3-methylpentanoate, propanoate, succinate, formic acid and pyruvate. The range of overflow metabolites produced in a particular fermentation can depend upon the limitation applied (e.g. nitrogen, phosphate, oxygen), the extent of the limitation, and the carbon source provided (Schlegel, H. G. & Vollbrecht, D. Journal of General Microbiology 1980 117:475-481; Steinbüchel, A. & Schlegel, H. G. Appl Microbiol Biotechnol 1989 31: 168; Vollbrecht et al. Eur J Appl Microbiol Biotechnol 1978 6:145-155; Vollbrecht et al. European J. Appl. Microbiol. Biotechnol. 1979 7: 267; Vollbrecht, D. & Schlegel, H. G. European J. Appl. Microbiol. Biotechnol. 1978 6: 157; Vollbrecht, D. & Schlegel, H. G. European J. Appl. Microbiol. Biotechnol. 1979 7: 259).

In one aspect of the invention, applying a suitable nutrient limitation under defined fermentation conditions can thus result in an increase in the flux through a particular metabolic node. The application of this knowledge to C. necator strains genetically modified to produce desired chemical products via the same metabolic node can result in increased production of the desired product.

A cell retention strategy using a ceramic hollow fiber membrane can be employed to achieve and maintain a high cell density during fermentation. The principal carbon source fed to the fermentation can derive from a biological or non-biological feedstock. The biological feedstock can be, or can derive from, monosaccharides, disaccharides, lignocellulose, hemicellulose, cellulose, paper-pulp waste, black liquor, lignin, levulinic acid and formic acid, triglycerides, glycerol, glutamates, agricultural waste, thin stillage, condensed distillers' solubles or municipal waste such as fruit peel/pulp. The non-biological feedstock can be, or can derive from, natural gas, syngas, CO₂/H₂, CO, H₂, O₂, methanol, ethanol, non-volatile residue (NVR) a caustic wash waste stream from cyclohexane oxidation processes or waste stream from a chemical industry such as, but not limited to a carbon black industry or a hydrogen-refining industry, or petrochemical industry, a nonlimiting example being a PTA-waste stream.

In one nonlimiting embodiment, at least one of the enzymatic conversions of the production method comprises gas fermentation within the altered Cupriavidus necator host, or a member of the genera Ralstonia, Wautersia, Alcaligenes, Burkholderia and Pandoraea, and other organism having one or more of the above-mentioned properties of Cupriavidus necator. In this embodiment, the gas fermentation may comprise at least one of natural gas, syngas, CO₂/H₂, CO, H₂, O₂, methanol, ethanol, non-volatile residue, caustic wash from cyclohexane oxidation processes, or waste stream from a chemical industry such as, but not limited to a carbon black industry or a hydrogen-refining industry, or petrochemical industry. In one nonlimiting embodiment, the gas fermentation comprises CO₂/H₂.

The methods of the present invention may further comprise recovering produced compounds involved in glutamate metabolism or derivatives or compounds related thereto. Once produced, any method can be used to isolate the compound or compounds involved in glutamate metabolism or derivatives or compounds related thereto.

The present invention also provides altered organisms capable of biosynthesizing increased amounts of compounds involved in glutamate metabolism and derivatives and compounds related thereto as compared to the unaltered organism. In one nonlimiting embodiment, the altered organism of the present invention is a genetically engineered strain of Cupriavidus necator capable of producing compounds involved in glutamate metabolism and derivatives and compounds related thereto. In another nonlimiting embodiment, the organism to be altered is selected from members of the genera Ralstonia, Wautersia, Alcaligenes, Cupriavidus, Burkholderia and Pandoraea, and other organisms having one or more of the above-mentioned properties of Cupriavidus necator. In one nonlimiting embodiment, the present invention relates to a substantially pure culture of the altered organism capable of producing compounds involved in glutamate metabolism and derivatives and compounds related thereto.

As used herein, a “substantially pure culture” of an altered organism is a culture of that microorganism in which less than about 40% (i.e., less than about 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the altered microorganism, e.g., bacterial, fungal (including yeast), mycoplasmal, or protozoan cells. The term “about” in this context means that the relevant percentage can be 15% of the specified percentage above or below the specified percentage. Thus, for example, about 20% can be 17% to 23%. Such a culture of altered microorganisms includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen st orage or transport medium. The cultures are in a culture vessel or storage vessel or substrate (e.g., a culture dish, flask, or tube or a storage vial or tube).

Altered organisms of the present invention comprise introduction of at least one synthetic gene encoding one or multiple enzymes.

In one nonlimiting embodiment, the altered organism is produced by introduction of at least one synthetic gene encoding one or multiple enzymes thus redirecting the organism to produce compounds involved in glutamate metabolism, as well as derivatives and compounds related thereto.

In one nonlimiting embodiment, the organism is altered to express a GDC. In one nonlimiting embodiment, the GDC is from E. coli or B. megaterium. In one nonlimiting embodiment, the GDC comprises SEQ ID NO:2 or 4 or a polypeptide with similar enzymatic activities exhibiting at least about 50%, 60%, 70%, 75%, 80%, 85%, 90%, 910, 92%, 93%, 94%, 95%, 96%, 97%, 98%, 99% or 99.5% sequence identity to an amino acid sequence set forth in SEQ ID NO:2 or 4 or a functional fragment thereof. In one nonlimiting embodiment, the GDC is encoded by a nucleic acid sequence comprising SEQ ID NO:1 or 3 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO: 1 or 3 or a functional fragment thereof.

In one nonlimiting embodiment, the organism is altered to express or overexpress one or more enzymes such as, but not limited to, isocitrate dehydrogenase, glutamate dehydrogenase and glutamate synthase.

In one nonlimiting embodiment, the isocitrate dehydrogenase is from E. coli or C. glutamicum. In one nonlimiting embodiment, the isocitrate dehydrogenase comprises SEQ ID NO:13 or 15 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:13 or 15 or a functional fragment thereof. In one nonlimiting embodiment, the isocitrate dehydrogenase is encoded by a nucleic acid sequence comprising SEQ ID NO:12 or 14 or a nucleic acid sequence encoding a polypeptide with similar enzymatic activities exhibiting at least about 50%, 60%, 70%, 75%, 80%, 85%, 90%, 91%, 920, 93%, 94%, 95%, 96%, 97%, 98%, 99% or 99.5% sequence identity to the nucleic acid sequence set forth in SEQ ID NO: 12 or 14 or a functional fragment thereof.

In one nonlimiting embodiment, the glutamate dehydrogenase is from E. coli or C. necator. In one nonlimiting embodiment, the glutamate dehydrogenase comprises SEQ ID NO:9 or 11 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:9 or 11 or a functional fragment thereof. In one nonlimiting embodiment, the glutamate dehydrogenase is encoded by a nucleic acid sequence comprising SEQ ID NO:8 or 10 or a nucleic acid sequence encoding a polypeptide with similar enzymatic activities exhibiting at least about 50%, 60%, 70%, 75%, 80%, 85%, 90%, 91%, 92%, 93%, 94%, 950, 96%, 97%, 980, 99% or 99.5% sequence identity to the nucleic acid sequence set forth in SEQ ID NO: 8 or 10 or a functional fragment thereof.

In one nonlimiting embodiment, the glutamate synthase is from E. coli. In one nonlimiting embodiment, the glutamate synthase comprises SEQ ID NO:6 or 7 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:6 or 7 or a functional fragment thereof. In one nonlimiting embodiment, the glutamate synthase is encoded by a nucleic acid sequence comprising SEQ ID NO:5 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:5 or a functional fragment thereof.

In one nonlimiting embodiment, the organism is altered to express a GABA antiporter. In one nonlimiting embodiment, the GABA antiporter is from E. coli. In one nonlimiting embodiment, the GABA antiporter comprises SEQ ID NO:17 or a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to an amino acid sequence set forth in SEQ ID NO:17 or a functional fragment thereof. In one nonlimiting embodiment, the GABA antiporter is encoded by a nucleic acid sequence comprising SEQ ID NO:16 or a nucleic acid sequence encoding a polypeptide with similar enzymatic activities exhibiting at least about 50%, 60%, 70%, 75%, 80%, 85%, 90%, 91%, 92%, 93%, 94%, 95%, 96%, 97%, 98%, 99% or 99.5% sequence identity to the nucleic acid sequence set forth in SEQ ID NO:16 or a functional fragment thereof.

The altered organism of the present invention may express one or more or all of the above-described enzymes.

In one nonlimiting embodiment, the nucleic acid sequence of the synthetic operon is codon optimized for C. necator.

In one nonlimiting embodiment, the organism is further altered by deleting one or more genes which encode enzymes which degrade GABA such as, but not limited to a gabT gene.

In one nonlimiting embodiment, the organism is further altered by redirecting carbon towards glutamate and deleting competing pathways. In one nonlimiting embodiment, OdhA and subunits thereof such as OdhB are deleted.

Organisms produced in accordance with the present invention may comprise one or more or all of the above-described alterations.

In one nonlimiting embodiment, the organism is further modified to eliminate phaCAB, involved in PHBs production and/or H16-A0006-9 encoding endonucleases thereby improving transformation efficiency.

The percent identity (and/or homology) between two amino acid sequences as disclosed herein can be determined as follows. First, the amino acid sequences are aligned using the BLAST 2 Sequences (B12seq) program from the stand-alone version of BLAST containing BLASTP version 2.0.14. This stand-alone version of BLAST can be obtained from the U.S. government's National Center for Biotechnology Information web site (www with the extension ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be followed for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 90.11, 90.12, 90.13, and 90.14 is rounded down to 90.1, while 90.15, 90.16, 90.17, 90.18, and 90.19 is rounded up to 90.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the polypeptides or nucleic acid sequences described herein can also be used in the methods and organisms disclosed herein. The term “functional fragment” as used herein refers to a peptide fragment of a polypeptide or a nucleic acid sequence fragment encoding a peptide fragment of a polypeptide that has at least 25% (e.g., at least: 30%; 40%; 50%; 60%; 70%; 75%; 80%; 85%; 90%; 95%; 98%; 99%; 100%; or even greater than 100%) of the activity of the corresponding mature, full-length, polypeptide. The functional fragment can generally, but not always, be comprised of a continuous region of the polypeptide, wherein the region has functional activity.

Functional fragments may range in length from about 10% up to 99% (inclusive of all percentages in between) of the original full-length sequence.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a nonconservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagluttanin (HA), glutathione-S-transferase (GST), or maltose binding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Endogenous genes of the organisms altered for use in the present invention also can be disrupted to prevent the formation of undesirable metabolites or prevent the loss of intermediates in the pathway through other enzymes acting on such intermediates. In one nonlimiting embodiment, the organism is altered by deleting one or more genes which encode enzymes which degrade GABA such as, but not limited to a gabT gene. In one nonlimiting embodiment, the organism is altered by redirecting carbon towards glutamate and deleting competing pathways. In one nonlimiting embodiment, OdhA and subunits thereof such as OdhB are deleted. In one nonlimiting embodiment, the organism is further modified to eliminate phaCAB, involved in PHBs production and/or H16-A0006-9 encoding endonucleases thereby improving transformation efficiency.

Thus, as described herein, altered organisms can include exogenous nucleic acids redirecting the organism toward glutamate metabolism. In one nonlimiting embodiment, the exogenous nucleic acid encodes a GDC. In one nonlimiting embodiment, the exogenous nucleic acid encodes one or more enzymes of the TCA cycle such as, but not limited to, glutamate synthase, glutamate dehydrogenase and isocitrate dehydrogenase. In one nonlimiting embodiment, the exogenous nucleic acid encodes a GABA antiporter.

The term “exogenous” as used herein with reference to a nucleic acid (or a protein) and an organism refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a an organism or host once utilized by or in the organism or host. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

The present invention also provides exogenous genetic molecules of the nonnaturally occurring organisms disclosed herein such as, but not limited to, codon optimized nucleic acid sequences, expression constructs and/or synthetic operons.

In one nonlimiting embodiment, the exogenous genetic molecule comprises a codon optimized nucleic acid sequence encoding a GDC as disclosed herein. In one nonlimiting embodiment, the exogenous genetic molecule comprises a codon optimized nucleic acid sequence encoding an enzyme in the TCA cycle such as, but not limited to, isocitrate dehydrogenase, glutamate dehydrogenase and glutamate synthase as disclosed herein. In one nonlimiting embodiment, the exogenous genetic molecule comprises a codon optimized nucleic acid sequence encoding a GABA antiporter as disclosed herein. Additional nonlimiting examples of exogenous genetic molecules include expression constructs and synthetic operons of one or more of GDC, isocitrate dehydrogenase, glutamate dehydrogenase glutamate synthase and/or GABA antiporter as disclosed herein.

Also provided by the present invention are compounds involved in glutamate metabolism and derivatives and compounds related thereto bioderived from an altered organism according to any of methods described herein.

Further, the present invention relates to means and processes for use of these means for biosynthesis of compounds involved in glutamate metabolism and/or derivative compounds and/or compounds related thereto. Nonlimiting examples of such means include altered organisms and exogenous genetic molecules as described herein as well as any of the molecules as depicted in FIG. 1.

Also provided by the present invention are synthetic molecular probes. In one nonlimiting embodiment, the synthetic molecular probe comprises a primer such as disclosed herein. In one nonlimiting embodiment, the synthetic molecular probe is labeled for detection. Examples of detectable labels include, but are not limited to, fluorophores, radioactive isotopes and detectable agents such as, but not limit to, biotin.

In addition, the present invention provides bio-derived, bio-based, or fermentation-derived products produced using the methods and/or altered organisms disclosed herein. In one nonlimiting embodiment, a bio-derived, bio-based or fermentation derived product is produced in accordance with the exemplary central metabolism depicted in FIG. 1. Examples of such products include, but are not limited to, compositions comprising at least one bio-derived, bio-based, or fermentation-derived compound or any combination thereof, as well as dietary supplements, molded substances, formulations and semi-solid or non-semi-solid streams comprising one or more of the bio-derived, bio-based, or fermentation-derived compounds or compositions, combinations or products thereof.

In one aspect of the present invention, to synthesize GABA, carbon is diverted out of the TCA cycle by conversion of α-ketoglutarate to glutamate. Glutamate is then decarboxylated by glutamate decarboxylase (GDC) to form GABA. Cupridavidus contains exemplary genes necessary for this biochemical pathway, except for GDC.

The ΔphaCABΔA0006-9 background of Cupriavidus necator H16 was used.

The organisms can be altered to include GDC from biological resources, such as E. coli or Bacillus megaterium (Liu et al. Biotechnology letters 2016 38(7):1107-1113). As Cupriavidus necator can use GABA as a nitrogen source (Mayer & Cook Journal of bacteriology 2009 191(19): 6052-6058) with H16_B0981 being annotated as the responsible gabT gene, the organisms can be further altered to delete this gene in a construct referred to as ΔphaCABΔA0006-9ΔgabT. Constructs with enzymes of the TCA cycle, NAD(P)H-dependent glutamate dehydrogenase and/or isocitrate dehydrogenase can also be used. Further, the Cupriavidus necator genome does not have an annotated GABA transporter, but may have a GABA permease to allow import (Mayer & Cook Journal of bacteriology 2009 191(19):6052-6058). Accordingly constructs with a GABA antiporter can be prepared.

Nonlimiting examples of the above described constructs prepared in accordance with the present invention are shown in Table 1.

TABLE 1

| | | | Glutamate | |
|---|---|---|---|---|
| | | | synthase/ | |
| | Glutamate | Isocitrate | Dehy- | |
| Vector | decarboxylase | dehydrogenase | drogenase | Antiporter |
| pBBR1 | E. coli gadB | E. coli idh | E. coli GltB, | E. coli |
| pBAD1A* | | | GltD | GadC |
| | B. megaterium | C. Glutamicum | E. coli ghdA | |
| | GAD | idh | | |
| | | | C. necator | |
| | | | ghdA1 | |

*The 1A vector is a derivative of pBBR1-MCS2 as disclosed in sciencedirect with the extension .com/science/article/pii/0378111995005841 of the world wide web altered to be compatible with the assembly technique.

In this strategy, GDC is designed to be inserted into the pBAD expression vector. The TCA genes can be assembled to follow the GDC in various combinations. GadC may be inserted at the end of the operon, leading to an operon of 1-4 genes. Potential combinations for assembled vectors for Route A are listed in Table 2. Expression was from pBAD promoter to allow for potential toxicity of the products and for safety according to the GMMRA.

TABLE 2

| Construct | GDC | Idh | Glt/GS | GadC |
|---|---|---|---|---|
| EV | Empty | | | |
| | vector | | | |
| A1 | ecGadB | | | |
| A2 | bmGAD | | | |
| A3c | ecGadB | ecIdh | cnGDHA1 | |
| A3f | ecGadB | cgIdh | cnGDHA1 | |
| A4c | bmGAD | ecIdh | cnGDHA1 | |
| A4f | bmGAD | cgIdh | cnGDHA1 | |
| A5 | ecGadB | | | ecGadC |
| A6 | bmGAD | | | ecGadC |
| A7c | ecGadB | ecIdh | cnGDHA1 | ecGadC |
| GABA-A7f | ecGadB | cgIdh | cnGDHA1 | ecGadC |

GABA production was detected in the altered organisms of the present invention. Inclusion of the glutamate-GABA antiporter improved GABA production and contributed to the highest producing strain. Glutamate was depleted in the media and GABA production was improved. Inclusion of the TCA overexpression also leads to an improvement over GDC alone. Inclusion of C. necator GDH also improved GABA production. Lower pH also improved production of GABA and use with acidic feedstocks may be advantageous.

The following section provides further illustration of the methods and materials of the present invention. These Examples are illustrative only and are not intended to limit the scope of the invention in any way.

### EXAMPLES

Sample Analysis Via LC-MS

Extracellular γ-amino butyric acid, arginine, glutamic acid and ornithine concentrations were determined by liquid chromatography-mass spectrometry (LC-MS). Fermentation broth containing samples were centrifuged and the supernatants were diluted between 10- and 100-fold, depending upon anticipated analyte concentration.

LC-MS was performed using an Agilent Technologies (Santa Clara, Calif., USA) 1290 Series Infinity HPLC system, coupled to an Agilent 6530 Series Q-TOF mass spectrometer. Manufacturer instructions were followed, using a BEH Amide UPLC column: 2.1 mm diameter×50 mm length×1.7 μm particle size (Waters, Milford, Mass., USA). External standard curves were used for quantitation. Calibration levels were constructed in a matrix-matched solution, typically the blank medium, diluted to the same level as the samples in acetonitrile. Concentrations were determined by interpolation of sample responses against the calibration curve.

Primers

Table 3 discloses primers used to produce constructs suitable for use with the present invention.

TABLE 3

| | | | SEQ |
|---|---|---|---|
| | | | ID |
| Primer Name | Sequence | Purpose | NO: |
| sacB rev | gcatgggcataaagttgcctttttaatc | sequence and screen from | 18 |
| | | sacB | |
| Ori-T fwd | cggtgatgccacgatcctcgccctgctggc | Sequence and screen from | 19 |
| | | OriT | |
| pBAD pr reverse | ggatccgctaatcttatggataaaaatgc | Sequence and screen from | 20 |
| | | pBAD | |
| pBAD F | agcattctgtaacaaagcg | Sequence and screen from | 21 |
| | | pBAD | |
| pBBR1-fwd | tgcaaggcgattaagttggg | check assembly of level 1 | 22 |
| | | constructs | |
| pBBR1-rev | aagcgcgcaattaaccctc | check assembly of level 1 | 23 |
| | | constructs | |
| insertn F | gaaactctggctcaccgacg | Check assembly of level 2 | 24 |
| | | constructs | |
| insertn R | gctgttcagggatttgcagc | Check assembly of level 2 | 25 |
| | | constructs | |
| OdhABdel RHA | atagtgagcgtcccatgatgcagcagtatc | OdhAB deletion and check | 26 |
| fwd | tgctggacctgtaattaacg | | |
| OdhABdel LHA | gaccgttaattacaggtccagcagatactg | OdhAB deletion and check | 27 |
| rev | ctgcatcatgggacgctcac | | |
| OdhABdel RHA | ggtatatgtgatgggttaaaaaggatcgat | OdhAB deletion and check | 28 |
| rev | gaacttgagcgcgccctcgttatcg | | |
| OdhABdel LHA | tacatcaccgacgagcaaggcaagaccgat | OdhAB deletion and check | 29 |
| fwd | ccgccgttgctgtcatcgttttctg | | |
| OdhABdel up | gcaccgaggcgcgcaccggtgcg | OdhAB deletion and check | 30 |
| fwd | | | |
| OdhABdel down | gaacgcgggcagcgcttccag | OdhAB deletion and check | 31 |
| rev | | | |
| check ecGDCbeta | atcggccaccatgttgacgc | check assembly of expression | 32 |
| rev | | constructs | |
| check ecGDCbeta | tctgcttcaagctgaaggac | check assembly of expression | 33 |
| fwd | | constructs | |
| check bmGDCbeta | atgttcttgtcgaaggactcg | check assembly of expression | 34 |
| rev | | constructs | |
| check bmGDCbeta | cggtgctcgcctggaagctg | check assembly of expression | 35 |
| fwd | | constructs | |
| check ecGS rev | gcccacggcgtagttcttcg | check assembly of expression | 36 |
| | | constructs | |
| check ecGS fwd | tggccttcggcttccgtccg | check assembly of expression | 37 |
| | | constructs | |
| check ecGDH rev | tcaccgaggggtggaaacgc | check assembly of expression | 38 |
| | | constructs | |
| check ecGDH fwd | cgggcaaggccgccaatgcc | check assembly of expression | 39 |
| | | constructs | |
| check cnGDH rev | tgggcgatcgtgccgttgtc | check assembly of expression | 40 |
| | | constructs | |
| check cnGDH fwd | ccggacgtgatcgccaacgc | check assembly of expression | 41 |
| | | constructs | |
| check ecIDH rev | cacctgggtggacttctcac | check assembly of expression | 42 |
| | | constructs | |
| check ecIDH fwd | atcagctggcccgcgaagag | check assembly of expression | 43 |
| | | constructs | |
| check cgIDH rev | tggcattatccggcagctcg | check assembly of expression | 44 |
| | | constructs | |
| check cgIDH fwd | cctcaatgaagagaagtcgc | check assembly of expression | 45 |
| | | constructs | |
| RBS fwd | atgtacGGTCTCAGGATAAAGGAGGTATA | Altering overhangs of | 46 |
| | TCGATG | fragments by PCR | |
| RBS fwd | atgtacGGTCTCATATGAAAGGAGGTATA | Altering overhangs of | 47 |
| | TCGATG | fragments by PCR | |
| RBS fwd | atgtacGGTCTCAATCGAAAGGAGGTATA | Altering overhangs of | 48 |
| | TCGATG | fragments by PCR | |
| -rrnBt1t2Ter | atgatcggtctctagtacaacgtaggaag | Altering overhangs of | 49 |
| rev | agtttg | fragments by PCR | |
| ter rrnBt1t2 | cgctctcctgagtaggacaaatc | general sequencing/assembly | 50 |
| fwd | | checks | |
| ecGS-end rev | atgatcggtctctatcctcacacttccag | Altering overhangs of | 51 |
| | ccaattc | fragments by PCR | |
| ecGDH-end rev | atgatcggtctctatcctcagatcacgcc | Altering overhangs of | 52 |
| | ctgcgc | fragments by PCR | |
| cnGDH-end rev | atgatcggtctctatcctcacgggtacag | Altering overhangs of | 53 |
| | gccgc | fragments by PCR | |
| SpeC-end rev | atgatcggtctctgccatcacttcaggac | Altering overhangs of | 54 |
| | gtagccg | fragments by PCR | |
| check GadC0918 | gaagggcaaggccaacacg | check assembly of expression | 55 |
| fwd | | constructs | |
| check GadC0918 | gtactcgtagacggccatc | check assembly of expression | 56 |
| rev | | constructs | |
| check SpeC0919 | gtggtgccgggcgaggtgtg | check assembly of expression | 57 |
| fwd | | constructs | |
| check SpeC0919 | gaacaccggcagatggaaac | check assembly of expression | 58 |
| rev | | constructs | |
| check hpRocF0920 | agagcttcaaggaccgtctg | check assembly of expression | 59 |
| fwd | | constructs | |
| check hpRocF0920 | catgcccttgatcacatcgc | check assembly of expression | 60 |
| rev | | constructs | |
| check bsRocF0921 | gtgggcggcatcagctacc | check assembly of expression | 61 |
| fwd | | constructs | |
| check bsRocF0921 | gatgtcgcccaggtcctcgac | check assembly of expression | 62 |
| rev | | constructs | |
| check cgmA0922 | tgatcatcgccctggtctgc | check assembly of expression | 63 |
| fwd | | constructs | |
| check cgmA0922 | aggggcagggccgtatacag | check assembly of expression | 64 |
| rev | | constructs | |
| check AdiA0923 | cttcccgggcttcgagcacg | check assembly of expression | 65 |
| fwd | | constructs | |
| check AdiA0923 | caggcgctccacggcgttac | check assembly of expression | 66 |
| rev | | constructs | |
| check SpeB0924 | tgaaggacctgaacatcgtc | check assembly of expression | 67 |
| fwd | | constructs | |
| check SpeB0924 | catgggcaggcgcaggaag | check assembly of expression | 68 |
| rev | | constructs | |
| seq ecGS0899 | caagaccggtgatggctgc | check sequence of amplified | 69 |
| FWD1 | | fragment | |
| seq ecGS0899 | atcaccggcaaccgccagtg | check sequence of amplified | 70 |
| FWD2 | | fragment | |
| seq ecGS0899 | gaagaggtgggctcgcgcga | check sequence of amplified | 71 |
| FWD3 | | fragment | |
| seq ecGS0899 | accagtcgctgcgctgcgac | check sequence of amplified | 72 |
| FWD4 | | fragment | |
| seq ecGS0899 | gaacgccgtgaacatcgc | check sequence of amplified | 73 |
| FWD5 | | fragment | |
| seq ecGS0899 | ctgggagctgggcctcgtg | check sequence of amplified | 74 |
| FWD6 | | fragment | |
| seq ecGS0899 | ctccctgtcgggctatatcg | check sequence of amplified | 75 |
| FWD7 | | fragment | |
| seq ecGS0899 | ctggagcaccttcgcgacg | check sequence of amplified | 76 |
| FWD8 | | fragment | |
| seq ecGS0899 | gcaagaaggtcgcgatcatcg | check sequence of amplified | 77 |
| FWD9 | | fragment | |
| seq ecgdh900 | cgtgatccagttccgcgtg | check sequence of amplified | 78 |
| FWD1 | | fragment | |
| seq ecgdh900 | cggtgcgcgcgtcattacc | check sequence of amplified | 79 |
| FWD2 | | fragment | |
| seq cnGDH901 | caagtcgatcgggtgacgc | check sequence of amplified | 80 |
| FWD1 | | fragment | |
| seq cnGDH901 | ccagggttttggcaacgtg | check sequence of amplified | 81 |
| FWD2 | | fragment | |
| seq GadC918 | gtaaggccaagcaactgacc | check sequence of amplified | 82 |
| fwd1 | | fragment | |
| seq GadC918 | cgttcatcctctcgtacatg | check sequence of amplified | 83 |
| fwd2 | | fragment | |
| seq GadC918 | cggcggcaagggcgtgaagc | check sequence of amplified | 84 |
| fwd3 | | fragment | |
| seq SpeC919 | ggccgcggtggtcatcacg | check sequence of amplified | 85 |
| fwd1 | | fragment | |
| seq SpeC919 | cggtctacctggaagcgag | check sequence of amplified | 86 |
| fwd2 | | fragment | |
| seq SpeC919 | gctgtttcggcccttcatcc | check sequence of amplified | 87 |
| fwd3 | | fragment | |
| Seq CgmA fwd1 | cccaccaagacgcagcgctg | check sequence of amplified | 88 |
| | | fragment | |
| Seq CgmA fwd2 | tccttctatgccctgctcac | check sequence of amplified | 89 |
| | | fragment | |
| Seq CgmA fwd3 | cgccggcctgttcttcatg | check sequence of amplified | 90 |
| | | fragment | |
| OdhABdel RHA | atgtacggtctcagtatctgctggac | OdhAB deletion and check | 91 |
| fwd GG | ctgtaattaacggt | | |
| OdhABdel LHA | gtacatggtctctatactgctgcatc | OdhAB deletion and check | 92 |
| rev GG | atgggac | | |
| OdhABdel RHA | gtacatggtctctagtagaacttgag | OdhAB deletion and check | 93 |
| rev GG | cgcgccctc | | |
| OdhABdel LHA | atgtacggtctcaatcgccgccgttg | OdhAB deletion and check | 94 |
| fwd GG | ctgtc | | |
| BsaI-I-RBS | atgtacGGTCTCATATGAAAGGAGGT | Altering overhangs of | 95 |
| atgctg fwd | ATATCGATGctg | fragments by PCR | |
| Odhdel fwd 2 | ctggacgcgcagtcgctgtg | OdhAB deletion and check | 96 |
| Odhdel int rev | gatatcccggccgttcgagc | OdhAB deletion and check | 97 |
| odhdel int fwd | tgggcctggtggccatgaag | OdhAB deletion and check | 98 |

Sequence Information for Sequences in Sequence Listing

TABLE 4

| SEQ | |
|---|---|
| ID NO: | Sequence Description |
| 1 | Nucleic acid sequence of ecGadB - Glutamate |
| | decarboxylase beta UniProtKB/Swiss-Prot: P69910.1 |
| | EC4.1.1.15 |
| 2 | Amino acid sequence of ecGadB |
| 3 | Nucleic acid sequence of BmGAD Glutamate decarboxylase |
| | beta Bacillus megaterium KT895523 EC4.1.1.15 |
| 4 | Amino acid sequence of BmGAD protein sequence |
| 5 | Nucleic acid sequence of ecGS Glutamate synthase [NADPH] |
| | large chain/small chain |
| 6 | Amino acid sequence of ecGS - large chain |
| 7 | Amino acid sequence of ecGS - short chain |
| 8 | Nucleic acid sequence of ecGdh Glutamate dehydrogenase |
| | P00370 EC1.3.1.4 |
| 9 | Amino acid sequence of ecGdh |
| 10 | Nucleic acid sequence of cnGdh Glutamate dehydrogenase |
| | Q0KEF0/H16₋ A0471 EC1.3.1.3 |
| 11 | Amino acid sequence of cnGdh |
| 12 | Nucleic acid sequence of ecIdh Isocitrate dehydrogenase |
| | P08200 1.1.1.42 |
| 13 | Amino acid sequence of ecIdh |
| 14 | Nucleic acid sequence of cgIdh Isocitrate dehydrogenase |
| | P50216 c glutamicum 1.1.1.42 |
| 15 | Amino acid sequence of cgIdh |
| 16 | Nucleic acid sequence of ecGadC Glutamate/GABA antiporter |
| | P63235 E. coli |
| 17 | Amino acid sequence of ecGadC |
| 18-98 | Primer depicted in Table 3 |

1. A process for the biosynthesis of compounds involved in glutamate metabolism, and/or derivatives thereof and/or compounds related thereto, said process comprising:
obtaining an organism capable of producing compounds involved in glutamate metabolism, derivatives thereof and/or compounds related thereto;
altering the organism; and
producing more compounds involved in glutamate metabolism, and/or derivatives thereof and/or compounds related thereto by the altered organism as compared to the unaltered organism.

2. The process of claim 1 wherein the organism is C. necator or an organism with properties similar thereto.

3. The process of claim 1 wherein the organism is altered to express a glutamate decarboxylase (GDC).

4. The process of claim 3 wherein the GDC is from E. coli or B. megaterium.

5. The process of claim 3 wherein the GDC comprises SEQ ID NO:2 or 4 or a polypeptide with similar enzymatic activities exhibiting at least about 50% sequence identity to an amino acid sequence set forth in SEQ ID NO:2 or 4 or a functional fragment thereof or is encoded by a nucleic acid sequence comprising SEQ ID NO:1 or 3 or a nucleic acid sequence encoding a polypeptide with similar enzymatic activities exhibiting at least about 50% sequence identity to the nucleic acid sequence set forth in SEQ ID NO: 1 or 3 or a functional fragment thereof.

6. (canceled)

7. The process of claim 1 wherein the organism is altered to express or overexpress one or more enzymes.

8. The process of claim 7 wherein the enzymes are selected from isocitrate dehydrogenase, glutamate dehydrogenase and glutamate synthase.

9. The process of claim 8 wherein the isocitrate dehydrogenase is from E. coli or C. glutamicum, the glutamate dehydrogenase is from E. coli or C. necator, and/or the glutamate synthase is from E. coli.

10. The process of claim 8 wherein the isocitrate dehydrogenase comprises SEQ ID NO:13 or 15 or a polypeptide with similar enzymatic activities exhibiting at least about 50% sequence identity to an amino acid sequence set forth in SEQ ID NO:13 or 15 or a functional fragment thereof or is encoded by a nucleic acid sequence comprising SEQ ID NO:12 or 14 or a nucleic acid sequence encoding a polypeptide with similar enzymatic activities exhibiting at least about 50% sequence identity to the nucleic acid sequence set forth in SEQ ID NO:12 or 14 or a functional fragment thereof.

11-12. (canceled)

13. The process of claim 8 wherein the glutamate dehydrogenase comprises SEQ ID NO:9 or 11 or a polypeptide with similar enzymatic activities exhibiting at least about 50% sequence identity to an amino acid sequence set forth in SEQ ID NO:9 or 11 or a functional fragment thereof or is encoded by a nucleic acid sequence comprising SEQ ID NO:8 or 10 or a nucleic acid sequence encoding a polypeptide with similar enzymatic activities exhibiting at least about 50% sequence identity to the nucleic acid sequence set forth in SEQ ID NO:8 or 10 or a functional fragment thereof.

14-15. (canceled)

16. The process of claim 8 wherein the glutamate synthase comprises SEQ ID NO:6 or 7 or a polypeptide with similar enzymatic activities exhibiting at least about 50% sequence identity to an amino acid sequence set forth in SEQ ID NO:6 or 7 or a functional fragment thereof or is encoded by a nucleic acid sequence comprising SEQ ID NO:5 or a nucleic acid sequence encoding a polypeptide with similar enzymatic activities exhibiting at least about 50% sequence identity to the nucleic acid sequence set forth in SEQ ID NO:5 or a functional fragment thereof.

17. (canceled)

18. The process of claim 1 wherein the organism is altered to express a GABA antiporter and/or by deleting one or more genes which encode enzymes which degrade GABA and/or by redirecting carbon towards glutamate and deleting competing pathways.

19. The process of claim 18 wherein the GABA antiporter is from E. coli.

20. The process of claim 18 wherein the GABA antiporter comprises SEQ ID NO:17 or a polypeptide with similar enzymatic activities exhibiting at least about 50% sequence identity to an amino acid sequence set forth in SEQ ID NO:17 or a functional fragment thereof or is encoded by a nucleic acid sequence comprising SEQ ID NO:16 or a nucleic acid sequence encoding a polypeptide with similar enzymatic activities exhibiting at least about 50% sequence identity to the nucleic acid sequence set forth in SEQ ID NO:16 or a functional fragment thereof.

21-22. (canceled)

23. The process of claim 18 wherein a gabT gene is deleted.

24. (canceled)

25. The process of claim 18 wherein OdhA and/or OdhB is deleted.

26-28. (canceled)

29. The process of claim 1 wherein the organism is further altered to eliminate phaCAB, involved in PHBs production and/or H16-A0006-9 encoding endonucleases thereby improving transformation efficiency.

30. (canceled)

31. An altered organism capable of producing more compounds involved in glutamate metabolism, derivatives thereof and/or compounds related thereto as compared to an unaltered organism.

32. The altered organism of claim 31 which is C. necator or an organism with properties similar thereto.

33. The altered organism of claim 31 which expresses a glutamate decarboxylase (GDC).

34. The altered organism of claim 33 wherein the GDC is from E. coli or B. megaterium.

35. The altered organism of claim 33 wherein the GDC comprises SEQ ID NO:2 or 4 or a polypeptide with similar enzymatic activities exhibiting at least about 50% sequence identity to an amino acid sequence set forth in SEQ ID NO: 2 or 4 or a functional fragment thereof or is encoded by a nucleic acid sequence comprising SEQ ID NO:1 or 3 or a nucleic acid sequence encoding a polypeptide with similar enzymatic activities exhibiting at least about 50% sequence identity to the nucleic acid sequence set forth in SEQ ID NO: 1 or 3 a functional fragment thereof.

36. (canceled)

37. The altered organism of claim 31 which expresses or overexpresses one or more enzymes.

38. The altered organism of claim 37 wherein the enzymes are selected from isocitrate dehydrogenase, glutamate dehydrogenase and glutamate synthase.

39. The altered organism of claim 37 wherein the isocitrate dehydrogenase is from E. coli or C. glutamicum and/or the glutamate dehydrogenase is from E. coli or C. necator and/or the glutamate synthase is from E. coli.

40. The altered organism of claim 38 wherein the isocitrate dehydrogenase comprises SEQ ID NO:13 or 15 or a polypeptide with similar enzymatic activities exhibiting at least about 50% sequence identity to an amino acid sequence set forth in SEQ ID NO:13 or 15 or a functional fragment thereof or is encoded by a nucleic acid sequence comprising SEQ ID NO:12 or 14 or a nucleic acid sequence encoding a polypeptide with similar enzymatic activities exhibiting at least about 50% sequence identity to the nucleic acid sequence set forth in SEQ ID NO:12 or 14 or a functional fragment thereof.

41-42. (canceled)

43. The altered organism of claim 38 wherein the glutamate dehydrogenase comprises SEQ ID NO:9 or 11 or a polypeptide with similar enzymatic activities exhibiting at least about 50% sequence identity to an amino acid sequence set forth in SEQ ID NO:9 or 11 or a functional fragment thereof or is encoded by a nucleic acid sequence comprising SEQ ID NO:8 or 10 or a nucleic acid sequence encoding a polypeptide with similar enzymatic activities exhibiting at least about 50% sequence identity to the nucleic acid sequence set forth in SEQ ID NO:8 or 10 or a functional fragment thereof.

44-45. (canceled)

46. The altered organism of claim 38 wherein the glutamate synthase comprises SEQ ID NO:6 or 7 or a polypeptide with similar enzymatic activities exhibiting at least about 50% sequence identity to an amino acid sequence set forth in SEQ ID NO:6 or 7 or a functional fragment thereof or is encoded by a nucleic acid sequence comprising SEQ ID NO:5 or a nucleic acid sequence encoding a polypeptide with similar enzymatic activities exhibiting at least about 50% sequence identity to the nucleic acid sequence set forth in SEQ ID NO:5 or a functional fragment thereof.

47. (canceled)

48. The altered organism of claim 31 which expresses a GABA antiporter, wherein one or more genes which encode enzymes which degrade GABA are deleted and/or wherein carbon is redirected towards glutamate by deleting competing pathways.

49. The altered organism of claim 48 wherein the GABA antiporter is from E. coli.

50. The altered organism of claim 48 wherein the GABA antiporter comprises SEQ ID NO:17 or a polypeptide with similar enzymatic activities exhibiting at least about 50% sequence identity to an amino acid sequence set forth in SEQ ID NO:17 or a functional fragment thereof or is encoded by a nucleic acid sequence comprising SEQ ID NO:16 or a nucleic acid sequence encoding a polypeptide with similar enzymatic activities exhibiting at least about 50% sequence identity to the nucleic acid sequence set forth in SEQ ID NO:16 or a functional fragment thereof.

51-52. (canceled)

53. The altered organism of claim 48 wherein a gabT gene is deleted.

54. (canceled)

55. The altered organism of claim 48 wherein OdhA and/or OdhB is deleted.

56-58. (canceled)

59. The altered organism of claim 31 wherein the organism is further altered to eliminate phaCAB, involved in PHBs production and/or H16-A0006-9 encoding endonucleases thereby improving transformation efficiency.

60. (canceled)

61. A bio-derived, bio-based, or fermentation-derived product produced from the method of claim 1, wherein said product comprises:
(i) a composition comprising at least one bio-derived, bio-based, or fermentation-derived compound or any combination thereof;
(ii) a bio-derived, bio-based, or fermentation-derived dietary supplement comprising the bio-derived, bio-based, or fermentation-derived composition or compound of (i), or any combination thereof;
(iii) a molded substance obtained by molding the bio-derived, bio-based, or fermentation-derived composition or compound of (i), or any combination thereof;
(iv) a bio-derived, bio-based, or fermentation-derived formulation comprising the bio-derived, bio-based, or fermentation-derived composition or compound of (i), the bio-derived, bio-based, or fermentation-derived diestery supplements of (ii), or the bio-derived, bio-based, or fermentation-derived molded substance of (iii), or any combination thereof; or
(v) a bio-derived, bio-based, or fermentation-derived semi-solid or a non-semi-solid stream, comprising the bio-derived, bio-based, or fermentation-derived composition or compound of (i), the bio-derived, bio-based, or fermentation-derived dietary supplements of (ii), the bio-derived, bio-based, or fermentation-derived formulation of (iii), or the bio-derived, bio-based, or fermentation-derived molded substance of (iv), or any combination thereof.

62. A bio-derived, bio-based or fermentation derived product produced in accordance with the central metabolism depicted in FIG. 1.

63. An exogenous genetic molecule of the altered organism of claim 31.

64. The exogenous genetic molecule of claim 63 comprising a codon optimized nucleic acid sequence or an expression construct or synthetic operon of one or more of GDC, isocitrate dehydrogenase, glutamate dehydrogenase glutamate synthase and/or GABA antiporter.

65. The exogenous genetic molecule of claim 63 codon optimized for C. necator.

66. The exogenous genetic molecule of claim 63 comprising a codon optimized nucleic acid sequence encoding a GDC, an enzyme in the TCA cycle or a GABA antiporter.

67.-70. (canceled)

71. A process for the biosynthesis of compounds involved in glutamate metabolism, derivatives thereof and/or compounds related thereto, said process comprising providing a means capable of producing compounds involved in glutamate metabolism, derivatives thereof and/or compounds related thereto, and producing compounds involved in glutamate metabolism, derivatives thereof and/or compounds related thereto with said means.

72. A synthetic molecular probe comprising a nucleic acid sequence as set forth in any of SEQ ID NOs 18-98.

73. A process for biosynthesis of compounds involved in glutamate metabolism, and derivatives thereof, and compounds related thereto, said process comprising:
a step for performing a function of altering an organism capable of producing compounds involved in glutamate metabolism, derivatives thereof, and/or compounds related thereto such that the altered organism produces more compounds involved in glutamate metabolism, derivatives thereof, and/or compounds compared to a corresponding unaltered organism; and
a step for performing a function of producing compounds involved in glutamate metabolism, derivatives thereof, and/or compounds related thereto in the altered organism.

74-75. (canceled)
